(19) United States
(12) Patent Application Publication
(10) Pub. No.: US 2025/0195567 A1
(43) Pub. Date: Jun. 19, 2025

(54) COMPOSITIONS FOR REMOVING MICROBIAL BIOFILM OR INHIBITING FORMATION THEREOF

(71) Applicants: Prudentix Ltd., Lod (IL); Hadasit Medical Research Services and Development Ltd., Jerusalem (IL)

(72) Inventors: Hagit SACKS, Modi'in (IL); Meir STERN, Rehovot (IL); David POLAK, Beit Hashmonai (IL); Igal LIAPIS, Vaughan (CA); David Dadi SEGAL, Tel-Aviv (IL); Danny ROSENBAUM, Kfar Saba (IL)

(21) Appl. No.: 18/846,130

(22) PCT Filed: Mar. 12, 2023
(86) PCT No.: PCT/IL2023/050253

§ 371 Date: Sep. 11, 2024

(30) Foreign Application Priority Data
Mar. 14, 2022 (IL) 291349

(51) Int. Cl. A61K 33/42; A61K 9/00; A61K 47/18; A61K 47/42; A61P 1/02

(52) U.S. Cl. CPC A61K 33/42; A61K 9/0063; A61K 47/183; A61K 47/42; A61P 1/02

(57) ABSTRACT

Compositions containing a polyphosphate and an amino acid N,N-diacetic acid, formulated as either liquid compositions that upon warming to body temperature solidify into a viscous gel, or solid dosage forms, which are useful as dental compositions for removing microbial biofilm, or inhibiting or disrupting formation thereof. A method for removing microbial biofilm from, or inhibiting or disrupting microbial biofilm formation in, a periodontal pocket, gingival pocket, pocket resulting from peri-implantitis, or caries associated with dental cavities or the root canal system, including administering the compositions.

### TECHNICAL FIELD

The present invention provides compositions comprising a polyphosphate and an amino acid N,N-diacetic acid, formulated as either liquid compositions that upon warming to body temperature solidify into a viscous gel; or solid dosage forms, which are useful as dental compositions for removing microbial biofilm, or inhibiting or disrupting formation thereof.

### BACKGROUND ART

A biofilm comprises a consortium of various microorganisms that coexist together and most often grow on a surface. These adherent microorganisms produce a slimy extracellular matrix of extracellular polymeric substances (EPSs) predominantly made from polysaccharides.

Biofilm mediated chronic infections are difficult, or impossible, to eliminate with conventional antibiotic and include otitis media, prostatitis, cystic fibrosis pneumonia, necrotising fasciitis, osteomyelitis, periodontitis, biliary tract infection, struvite kidney stone, and nosocomial infections.

Biofilms form on living or non-living surfaces and are prevalent in natural, industrial, and hospital settings. Since biofilms are resistant to antimicrobial agents, immune response and detergents, they pose a concern to public health.

Biofilms form on the teeth of all animals as dental plaque, where they potentially cause tooth decay and gum disease. Periodontitis refers to inflammation of the supporting tissues of the teeth with irreversible loss of the periodontal ligament attachment and bony support. This condition is characterized by periodontal pocket (a space between the teeth and the gums) formation and loss of attachment. With progression, tooth mobility and tooth loss emerge. It is estimated that nearly half of adults in the United States aged 30 years or older have some degree of periodontitis. The prevalence increases with age and is greater among males than females. Peri-implantitis is a destructive inflammatory process that affects the soft and hard tissues around an osseo-integrated dental implant. The soft tissues become inflamed whereas the alveolar bone (hard tissue), which surrounds the implant is lost over time. The reported prevalence of peri-implantitis varies from about 7% to 37% of implants. Periodontitis and peri-implantitis have similar biological and clinical characteristics. The treatment of both diseases today is inefficient and mainly based on mechanical removal of the bacterial plaque from the dental surface (procedure called scaling and root planing). Treatment may also include antibiotics although biofilm is reported to be resistant to it.

Polyphosphate is a negatively charged polymer, composed of many repeating units of orthophosphate linked by phosphoanhydride bonds, and can adopt linear or a cyclic ring structure. It is a sequestrant and forms chelate complexes with polyvalent metal ions. Sequestrants are a type of preservative, and polyphosphate is widely used as a food additive (E452i). In addition, polyphosphate is used in toothpastes as an anti-staining/anti-calculus and tartar prevention ingredient.

Recent findings indicate that orally administered polyphosphate suppresses biofilm production of P. aeruginosa and S. marcescens in animal models of intestinal injury (Hyoju et al., 2018).

Humphreys et al. (2011) show that ionic silver and polyphosphate (sodium hexametaphosphate), when combined, exhibit putative anti-biofilm synergy against P. aeruginosa, C. albicans and S. aureus, microorganisms associated with chronic wounds.

WO 2015/032447 discloses a detergent composition comprising an alkali metal carbonate, methylglycine-N,N-diacetic acid, glutamic acid N,N-diacetic acid, and alkali metal tripolyphosphate, said to be useful for removal of soil comprising oxidized polyphenols and calcium silicates, e.g., for removal of tea and coffee soil in ware washing applications.

JP 2020176274 discloses a composition comprising a chelating agent selected from alkali metal tripolyphosphates, MGDA, GLDA, and mixtures thereof, a percarbonate, and a peroxidation catalyst, for removal of tea and coffee stains in dishwashing applications.

CN 110860555 discloses a method for washing heavy metal polluted soil by mixing sodium hexametaphosphate with GLDA aqueous solution, to obtain an eluting agent which is then mixed with the polluted soil and eluted.

WO 2018/158764 discloses a liquid composition comprising a non-biodegradable thermosensitive polyalkylene oxide block copolymer, e.g., a poloxamer, a low molecular weight hyaluronic acid, and optionally a therapeutic agent, which solidifies into a viscous gel upon warming to body temperature and then releases said hyaluronic acid and said therapeutic agent, when present, in a sustained release manner.

### SUMMARY OF INVENTION

Disclosed herein are two medical compositions containing, as active agents, a polyphosphate (PolyP) or a salt thereof, and an amino acid N,N-diacetic acid or a salt thereof, wherein one of the compositions is in the form of a liquid at room temperature (16-25° C.) and/or under refrigerated conditions (2-8° C.), and upon warming to body temperature solidifies into a viscous gel; and the other composition is in the form of a solid dosage form, e.g., an essentially three-dimensional solid snippet (i.e., chip) adapted for implantation in a periodontal/peri-implant pocket, and upon contact with an aqueous fluid adsorbs said fluid, swells, and then degrades and releases said active agents in a sustained release manner.

As shown herein, these compositions, regardless of their specific formulation, are useful, e.g., as dental compositions, more specifically for removing microbial biofilm from, or inhibiting or disrupting microbial biofilm formation in, a periodontal pocket, gingival pocket, pocket resulting from peri-implantitis, or caries associated with dental cavities or the root canal system; and may further be used for removing microbial biofilm from, or inhibiting or disrupting microbial biofilm formation on, an orthodontic device such as orthodontic-brace, aligner, extender and bridge. Said compositions in fact offer treatment of gum disease and contaminated implants based on a potent chemical wash, which does not directly kill the bacteria but rather breaks down the spatial structure of the plaque and as a result, leads to flushing of the bacteria from the contaminated site.

In one aspect, the present invention thus provides a composition comprising, as active agents, at least one PolyP or a salt thereof, and a biodegradable chelating agent, more specifically an amino acid N,N-diacetic acid or a salt thereof, for use in removing microbial biofilm from, or inhibiting or disrupting microbial biofilm formation in, a periodontal pocket, gingival pocket, pocket resulting from peri-implantitis, caries associated with dental cavities or the root canal system, or orthodontic device such as orthodontic brace, aligner, extender and bridge. Particular such compositions are those wherein said PolyP is a polymetaphosphate and said amino acid N,N-diacetic acid is selected from glutamic acid-N,N-diacetic acid (GLDA), aspartic acid-N,N-diacetic acid, glycine-N,N-diacetic acid, methylglycine-N,N-diacetic acid (MGDA), serine-N,N-diacetic acid, and alpha- and beta-alanine-N,N-diacetic acid.

In one particular such aspect, the composition for use disclosed herein is formulated as liquid at room temperature and/or under refrigerated conditions, and upon warming to body temperature solidifies into a viscous gel. More specifically, such a composition (also referred to herein as “poloxamer copolymer-based composition”) further comprises a non-biodegradable thermosensitive pharmaceutically acceptable poloxamer copolymer, wherein the amount of said poloxamer copolymer in said composition is from about 17% to about 27% by weight, the amount of said polyphosphate or salt thereof in said composition is from about 0.05% to about 3% by weight, and the amount of said amino acid N,N-diacetic acid or salt thereof in said composition is from about 0.025% to about 2% by weight,
wherein said composition has a pH in a range of 6-8; and said composition is liquid at room temperature and/or under refrigerated conditions, and upon warming to body temperature, said composition solidifies into a viscous gel. Particular such compositions are those wherein said poloxamer copolymer is poloxamer 407, poloxamer 188, poloxamer 124, poloxamer 237, poloxamer 338, or a mixture thereof, e.g., poloxamer 407.

In another particular such aspect, the composition for use disclosed herein is formulated as a solid dosage form, e.g., an essentially three-dimensional solid implant adapted for implantation in a periodontal/peri-implant pocket, and upon contact with an aqueous fluid adsorbs said fluid, swells, and then degrades and releases said active agents in a sustained release manner. More specifically, such a composition (also referred to herein as “crosslinked polymer-based composition”) further comprises a water insoluble biodegradable or bioerodible pharmaceutically acceptable crosslinked polymer and a plasticizer, wherein the amount of said polymer in said composition is from about 50% to about 80% by weight, the amount of said plasticizer in said composition is from about 8% to about 13% by weight, the amount of said polyphosphate or salt thereof in said composition is from about 0.5% to about 25%, preferably from about 4% to about 25%, by weight, and the amount of said amino acid N,N-diacetic acid or salt thereof in said composition is from about 0.5% to about 10%, preferably from about 0.5% to about 8% by weight,
wherein said composition being in a solid dosage form, and upon contact with an aqueous fluid, said composition adsorbs said fluid and consequently swells, and then degrades and releases said polyphosphate or salt thereof and said amino acid N,N-diacetic acid or salt thereof in a sustained release manner. Particular such compositions are those wherein said polymer is a protein, e.g., gelatin preferably hydrolyzed, and said plasticizer is a phthalate ester, a phosphate ester, glycerin, or sorbitol.

In another aspect, the present invention relates to a method for removing microbial biofilm from, or inhibiting or disrupting microbial biofilm formation in, a periodontal pocket, gingival pocket, pocket resulting from peri-implantitis, or caries associated with dental cavities or the root canal system, in a subject in need thereof, comprising administering into said periodontal pocket, gingival pocket, pocket resulting from peri-implantitis, or caries associated with dental cavities or the root canal system, a composition as referred to above, e.g., a poloxamer copolymer-based composition or a crosslinked polymer-based composition, to thereby release said polyphosphate or salt thereof and said amino acid N,N-diacetic acid or salt thereof in said periodontal pocket, gingival pocket, pocket resulting from peri-implantitis, or caries associated with dental cavities or the root canal system, in a sustained, i.e., prolonged, release manner. In certain embodiments, the method disclosed is for removing microbial biofilm from, or inhibiting or disrupting microbial biofilm formation in, a periodontal pocket, gingival pocket, or pocket resulting from peri-implantitis, and comprises (a) topically administering into said periodontal pocket, gingival pocket, or pocket resulting from peri-implantitis, a poloxamer copolymer-based composition as defined above; or (b) implanting in said periodontal pocket, gingival pocket, or pocket resulting from peri-implantitis, a crosslinked polymer-based composition as defined above, to thereby release said polyphosphate or salt thereof and said amino acid N,N-diacetic acid or salt thereof in said periodontal pocket, gingival pocket, or pocket resulting from peri-implantitis in a prolonged release manner. In other embodiments, the method disclosed is for removing microbial biofilm from, or inhibiting or disrupting microbial biofilm formation in, caries associated with dental cavities or the root canal system, and comprises topically administering into said caries associated with dental cavities or the root canal system a poloxamer copolymer-based composition as defined above, to thereby release said polyphosphate or salt thereof and said amino acid N,N-diacetic acid or salt thereof in said caries associated with dental cavities or the root canal system in a prolonged release manner.

In a further aspect, the present invention relates to a method for removing microbial biofilm from, or inhibiting or disrupting microbial biofilm formation on, an orthodontic device such as orthodontic-brace, aligner, extender and bridge, comprising administering onto said orthodontic device a composition as referred to above, e.g., a poloxamer copolymer-based composition, to thereby release said polyphosphate or salt thereof and said amino acid N,N-diacetic acid or salt thereof on said orthodontic device in a sustained release manner.

In still another aspect, the present invention provides a kit comprising a poloxamer copolymer-based composition as defined above, and a delivery mean, e.g., a syringe or an applicator, for topically administering or applying said composition into a periodontal pocket, gingival pocket, pocket resulting from peri-implantitis, or caries associated with dental cavities or the root canal system.

In yet another aspect, the present invention provides a kit comprising more than one solid dosage form as defined above, each being an essentially three-dimensional solid implant adapted for implantation in a periodontal pocket, for implanting in a periodontal pocket, gingival pocket, or pocket resulting from peri-implantitis.

In still a further aspect, the present invention provides a poloxamer copolymer-based composition as defined above per se, i.e., a composition comprising at least one PolyP or a salt thereof, an amino acid N,N-diacetic acid or a salt thereof, and a non-biodegradable thermosensitive pharmaceutically acceptable poloxamer copolymer, wherein the amount of said poloxamer copolymer in said composition is from about 17% to about 27% by weight, the amount of said PolyP or salt thereof in said composition is from about 0.05% to about 3% by weight, and the amount of said amino acid N,N-diacetic acid or salt thereof in said composition is from about 0.025% to about 2% by weight, wherein said composition has a pH in a range of 6-8; and said composition is liquid at room temperature and/or under refrigerated conditions, and upon warming to body temperature, said composition solidifies into a viscous gel.

In yet a further aspect, the present invention provides a crosslinked polymer-based composition as defined above per se, i.e., a composition comprising at least one PolyP or a salt thereof, an amino acid N,N-diacetic acid or a salt thereof, a water insoluble biodegradable or bioerodible pharmaceutically acceptable crosslinked polymer, and a plasticizer, wherein the amount of said crosslinked polymer in said composition is from about 50% to about 80% by weight, the amount of said plasticizer in said composition is from about 8% to about 13% by weight, the amount of said PolyP or salt thereof in said composition is from about 0.5% to about 25%, preferably from about 4% to about 25%, by weight, and the amount of said amino acid N,N-diacetic acid or salt thereof in said composition is from about 0.6% to about 10%, preferably from about 0.5% to about 8% by weight, wherein said composition being in a solid dosage form, and upon contact with an aqueous fluid, said composition adsorbs said fluid and consequently swells, and then degrades and releases said PolyP or salt thereof and said amino acid N,N-diacetic acid or salt thereof in a sustained release manner.

### BRIEF DESCRIPTION OF DRAWINGS

FIG. 1 shows microscopic images of biofilm grown on hydroxyapatite disks, and treated with saline (control) or saline with different concentration of PolyP (8%-0.2%). The biofilm that remained on the disks were stained with live/dead staining (green for live bacteria and red for dead bacteria).

FIG. 2 shows quantification of the microscopic results of the hydroxyapatite disks treated with PolyP as shown in FIG. 1. The results are expressed as mean and SD of fluorescent intensity (in RFU). Statistically significant results are marked with lines. *** indicates a statistical difference of p<0.001.

FIG. 3 shows quantification of the microscopic results of the titanium disks treated with PolyP, similar to the assay shown in FIG. 1. The results are expressed as mean and SD of fluorescent intensity (in RFU).

FIG. 4 shows quantification of the microscopic results of the hydroxyapatite disks treated with PolyP and GLDA at different concentrations. The results are expressed as mean and SD of fluorescent intensity (in RFU).

FIG. 5 shows quantification of the microscopic results of the titanium disks treated with PolyP and GLDA at different concentrations. The results are expressed as mean and SD of fluorescent intensity (in RFU).

FIG. 6 shows the detailed steps in the diffusion assay described in Example 6.

FIG. 7 shows quantification of the diffusion radius of poloxamer on hydroxyapatite disks treated with PolyP and GLDA at different concentrations (see Example 6). The results are expressed as mean and SD of halo distance.

FIG. 8 shows quantification of the diffusion radius of poloxamer on biofilm grown on titanium disks and treated with PolyP and GLDA at different concentrations. The results are expressed as mean and SD of halo distance.

FIG. 9 shows quantification of the diffusion radius of a snippet (chip) on biofilm grown on hydroxyapatite disks and treated with PolyP and GLDA at different concentrations. The results are expressed as mean and SD of halo distance.

FIG. 10 shows quantification of the diffusion radius of snippet (chip) on biofilm grown on titanium disks and treated with PolyP and GLDA at different concentrations. The results are expressed as mean and SD of halo distance.

FIG. 11 shows the procedure for establishing a pig model with implants and teeth. The steps specifically shown, illustrated in panels a-f, are incision in the gums; tissue elevation to expose bone; drilling in the bone; insertion of dental implant; screwing a golden shaded healing cap; and suturing with resorbable string.

FIG. 12 shows induction of periodontitis and periimplantitis in the pig model, using infected ligatures. Panels a-c—ligature binding around teeth and implants; panel b—after 8 weeks, ligatures were removed; panel c—x-ray demonstrating the implants; panel d—gingival crevicular fluid (GCF) was sampled using paper points; panel e—GCF was sampled using a perio strips; panel f—the sites were treated as detailed in the design ((1) gel test treatment; (2) gel sham treatment; (3) film test treatment 4; or (4) film sham treatment).

FIGS. 13A-13D show GCF's total protein and IL6 levels in teeth (13A and 13B, respectively) and implant (13C and 13D, respectively) before treatment, i.e., immediately after ligature removal (baseline) and 8 weeks post treatment. * and ** indicate statistically significant differences.

FIG. 14 shows pocket microbiome profile before (pre-treatment) and after treatment with the gel PolyP-GLDA in teeth. The size of the pie represents the total microbial load at the pocket, while the slices represent the relative abundance (in %) of the four bacteria that were immersed in the silk ligature during pocket formation.

FIG. 15 shows pocket microbiome profile before (pre-treatment) and after treatment with the film PolyP-GLDA in teeth. The size of the pie represents the total microbial load at the pocket, while the slices represent the relative abundance (in %) of the four bacteria immersed in the silk ligature during pocket formation.

FIG. 16 shows pocket microbiome profile before (pre-treatment) and after treatment with the gel PolyP-GLDA in implants. The size of the pie represents the total microbial load at the pocket, while the slices represent the relative abundance (in %) of the four bacteria immersed in the silk ligature during pocket formation.

FIG. 17 shows microbiome profile before (pre-treatment) and after treatment with the film PolyP-GLDA in implants. The size of the pie represents the total microbial load at the pocket, while the slices represent the relative abundance (in %) of the four bacteria immersed in the silk ligature during pocket formation.

FIG. 18A-18B show histological analysis of sites adjacent to teeth that were treated with test gel (18A) or sham gel (18B), indicating healthy gum tissue without evidence of inflammation or adverse tissue reaction.

### DETAILED DESCRIPTION

In one aspect, the present invention provides a composition as defined above, more specifically a medical composition comprising, as active agents, at least one polyphosphate (PolyP), or a salt thereof, and a biodegradable chelating agent, more specifically an amino acid N,N-diacetic acid or a salt thereof, for use in removing microbial biofilm from, or inhibiting or disrupting microbial biofilm formation in, a periodontal pocket, gingival pocket, pocket resulting from peri-implantitis, caries associated with dental cavities or the root canal system, or orthodontic device such as orthodontic brace, orthodontic aligner, orthodontic extender and orthodontic bridge.

The term “polyphosphate” or “polyphosphates” as used herein interchangeably refers to a highly anionic inorganic polymer, composed of orthophosphate monomers connected by high-energy phosphoanhydride bonds, which may have either linear or cyclic structure. The polyphosphate comprised within the composition of the present invention is preferably non-hydrolyzed polyphosphate. Yet, it should be clear that said composition may comprise a certain percentage of hydrolyzed polyphosphate or orthophosphate monomers as well, either present in the raw material (polyphosphate) utilized for the preparation of the composition, or due to degradation.

According to the present invention, the polyphosphate comprised within the composition disclosed herein may be in the form of a salt or a mixture thereof. Examples of polyphosphate salts include, without limiting, alkali metal salts such as sodium- or potassium salts of polyphosphate; alkaline earth metal salts such as magnesium- or calcium salts of polyphosphate; and ammonium salts of polyphosphate. In certain embodiments, the polyphosphate comprised within the composition is in the form of a sodium salt.

In certain embodiments, the polyphosphate comprised within the composition disclosed herein is a polymetaphosphate salt such as sodium polymetaphosphate, i.e., a compound of the formula (NaPO₃)ₙ wherein n is an integer of at least 2 and up to, e.g., 100, or a mixture of such compounds each having a different “n”. Examples of sodium polymetaphosphates include, without being limited to, mixtures of compounds each of the formula (NaPO₃)ₙ, wherein n each independently is in the range of 4 to 100, e.g., up to 10, 20, 30, 40, 50, 60, 70, 80, 90, or 100. In certain particular such embodiments, the term polymetaphosphate salt represents a mixture of compounds each of the formula (NaPO₃)ₙ, wherein said mixture comprises sodium hexametaphosphate, i.e., a compound of the formula (NaPO₃)₆ (also known as Calgon S, Graham's salt, or hexasodium metaphosphate). In other particular such embodiments, the term polymetaphosphate salt represents a mixture of compounds as defined above, either consisting or essentially consisting of sodium hexametaphosphate.

The term “chelating agent” generally refers to a chemical compound capable of forming one or more coordination or ionic bonds with metal ions to form stable, water-soluble, reversible metal complexes, and as used herein denotes an amino acid N,N-diacetic acid or a salt thereof. The term “amino acid” as used herein refers to an organic compound comprising both amine and carboxylic acid functional groups, which may be either a natural or non-natural amino acid and occur in both L and D isomeric forms. The twenty-two amino acids naturally occurring in proteins are aspartic acid, tyrosine, leucine, tryptophan, arginine, valine, glutamic acid, methionine, phenylalanine, serine, alanine, glutamine, glycine, proline, threonine, asparagine, lysine, histidine, isoleucine, cysteine, selenocysteine, and pyrrolysine. Non-limiting examples of other amino acids include citrulline, diaminopropionic acid, diaminobutyric acid, ornithine, aminoadipic acid, β-alanine, 1-naphthylalanine, 3-(1-naphthyl) alanine, 3-(2-naphthyl) alanine, γ-aminobutiric acid, 3-(aminomethyl)benzoic acid, p-ethynyl-phenylalanine, m-ethynyl-phenylalanine, p-chlorophenylalanine, p-bromophenylalanine, p-iodophenylalanine, p-acetylphenylalanine, p-azidophenylalanine, p-propargly-oxy-phenylalanine, indanylglycine, (benzyl) cysteine, norleucine, azidonorleucine, 6-ethynyl-tryptophan, 5-ethynyl-tryptophan, 3-(6-chloroindolyl) alanine, 3-(6-bromoindolyl) alanine, 3-(5-bromoindolyl) alanine, azidohomoalanine, α-aminocaprylic acid, O-methyl-L-tyrosine, N-acetylgalactosamine-α-threonine, and N-acetylgalactosamine-α-serine. Particular such chelating agents that may be comprised within the composition of the invention include, without being limited to, glutamic acid-N,N-diacetic acid (GLDA), aspartic acid-N,N-diacetic acid, glycine-N,N-diacetic acid, methylglycine-N,N-diacetic acid (MGDA), serine-N,N-diacetic acid, and alpha-alanine-N,N-diacetic acid, and beta-alanine-N,N-diacetic acid. In particular embodiments, the chelating agent comprised within the composition disclosed herein is GLDA, or a salt thereof such as tetrasodium glutamate diacetate (tetrasodium;(2S)-2-[bis(carboxylatomethyl)amino]pentanedioate).

In certain embodiments, the ratio between the polyphosphate or salt thereof and the amino acid N,N-diacetic acid or salt thereof, comprised within the composition disclosed herein, according to any one of the embodiments above, is from about 10:1 to about 1:6, e.g., from about 9:1 to about 1:5, from about 8:1 to about 1:4, from about 7:1 to about 1:3, from about 6:1 to about 1:2, or from about 5:1 to about 1:1, from about 4:1 to about 1:1, from about 3:1 to about 1:1, or from about 2:1 to about 1:1, preferably from about 5:1 to about 1:3, respectively, by weight.

In one particular such aspect, the composition disclosed herein is a poloxamer copolymer-based composition, i.e., a composition according to any one of the embodiments above, which further comprises a non-biodegradable thermosensitive pharmaceutically acceptable poloxamer copolymer, wherein the amount of said poloxamer copolymer in said composition is from about 17% to about 27%, e.g., 17-18%, 18-19%, 19-20%, 20-21%, 21-22%, 22-23%, 23-24%, 24-25%, 25-26%, or 26-27%, by weight, the amount of said polyphosphate or salt thereof in said composition is from about 0.05% to about 3%, e.g., 0.05-0.1%, 0.1-0.2%, 0.2-0.3%, 0.3-0.4%, 0.4-0.5%, 0.5-0.6%, 0.6-0.7%, 0.7-0.8%, 0.8-0.9%, 0.9-1%, 1-1.1%, 1.1-1.2%, 1.2-1.3%, 1.3-1.4%, 1.4-1.5%, 1.5-1.6%, 1.6-1.7%, 1.7-1.8%, 1.8-1.9%, 1.9-2%, 2-2.1%, 2.1-2.2%, 2.2-2.3%, 2.3-2.4%, 2.4-2.5%, 2.5-2.6%, 2.6-2.7%, 2.7-2.8%, 2.8-2.9%, or 2.9-3%, by weight, and the amount of said amino acid N,N-diacetic acid or salt thereof in said composition is from about 0.025% to about 2%, e.g., 0.05-0.1%, 0.1-0.2%, 0.2-0.3%, 0.3-0.4%, 0.4-0.5%, 0.5-0.6%, 0.6-0.7%, 0.7-0.8%, 0.8-0.9%, 0.9-1%, 1-1.1%, 1.1-1.2%, 1.2-1.3%, 1.3-1.4%, 1.4-1.5%, 1.5-1.6%, 1.6-1.7%, 1.7-1.8%, 1.8-1.9%, or 1.9-2%, by weight, wherein said composition has a pH in a range of about 6 to about 8; and said composition is liquid at room temperature and/or under refrigerated conditions, and upon warming to body temperature, said composition solidifies into a viscous gel.

Poloxamers have been widely used in the biomedical field due to their ability to undergo phase reverse thermal gelation. Their self-assembling process occurs through micellization, which is characterized by their critical micellization concentration and critical micellization temperature. These parameters, which depend on the specific poloxamer used and its concentration, as well as on the excipients added to the poloxamer and the concentration thereof, can be tailored to obtain materials with final properties suitable for a wide range of applications. Poloxamer gels are “generally regarded as safe” (GRAS) excipients and have been widely investigated and used for delivery of active agents. One of the drawbacks associated with poloxamer gels for delivery applications is short residence times due to lack of adhesiveness. Blending of poloxamers with mucoadhesive polymers that are capable of forming entanglements or non-covalent bonds with the mucus covering epithelial tissues is therefore one of the approaches to improve adhesiveness and residence time.

The term “poloxamer copolymer” as used herein denotes a polyethoxy/polypropoxy block copolymer, i.e., a nonionic triblock copolymer composed of a central hydrophobic chain of polyoxypropylene (poly(propylene oxide)) flanked by two hydrophilic chains of polyoxyethylene (poly(ethylene oxide)). Particular examples of such poloxamers include, without being limited to, poloxamer 407, poloxamer 188, poloxamer 124, poloxamer 237, poloxamer 338, or a mixture thereof. In particular embodiments, the poloxamer comprised within the poloxamer copolymer-based composition of the invention is poloxamer 407.

The present invention does not require the use of poloxamer copolymers having a specific level of purity, and thus polymers of any grade of purity may be employed. Moreover, the medical composition disclosed herein may contain more than one thermosensitive pharmaceutically acceptable polymer. It is, however, preferable to employ polymers having a high degree of purity, and especially a defined (i.e., specifiable) composition, since the use of such polymers increases the degree with which the release of the active agents, i.e., said polyphosphate or salt thereof and said biodegradable chelating agent (also referred to herein as “therapeutic agents”), may be controlled.

In certain embodiments, the composition disclosed herein is a poloxamer copolymer-based composition, wherein the poloxamer copolymer is poloxamer 407, poloxamer 188, poloxamer 124, poloxamer 237, poloxamer 338, or a mixture thereof. Particular such compositions are those wherein said poloxamer copolymer is poloxamer 407.

In certain embodiments, the composition disclosed herein is a poloxamer copolymer-based composition, wherein the polyphosphate is polymetaphosphate or a salt thereof such as sodium polymetaphosphate, and said amino acid N,N-diacetic acid is GLDA or a salt thereof such as tetrasodium glutamate diacetate. In particular such embodiments, said polymetaphosphate represents a compound of the formula (NaPO₃)ₙ wherein n is an integer of at least 2 and up to, e.g., 100, or a mixture thereof, comprising, consisting of, or essentially consisting of, hexametaphosphate. More particular such compositions are those comprising sodium polymetaphosphate in an amount of from about 0.1% to about 0.8%, e.g., about 0.1%, 0.15%, 0.2%, 0.25%, 0.3%, 0.35%, 0.4%, 0.45%, 0.5%, 0.55%, 0.6%, 0.65%, 0.7%, 0.75%, or 0.8%, by weight; tetrasodium glutamate diacetate in an amount of from about 0.025% to about 0.8%, e.g., about 0.05%, 0.1%, 0.15%, 0.2%, 0.25%, 0.3%, 0.35%, 0.4%, 0.45%, 0.5%, 0.55%, 0.6%, 0.65%, 0.7%, 0.75%, or 0.8%, by weight; and poloxamer 407 in an amount of from about 17% to about 26%, e.g., about 17%, 17.5%, 18%, 18.5%, 19%, 19.5%, 20%, 20.5%, 21%, 21.5%, 22%, 22.5%, 23%, 23.5%, 24%, 24.5%, 25%, 25.5%, or 26%, by weight, wherein each specific combination of said three ingredients represents a separate embodiment.

In specific embodiments, the composition disclosed herein is a poloxamer copolymer-based composition comprising: (i) sodium polymetaphosphate in an amount of about 0.2% by weight, tetrasodium glutamate diacetate in an amount of about 0.5% by weight, and poloxamer 407 in an amount of about 22-24% by weight; (ii) sodium polymetaphosphate in an amount of about 0.5% by weight, tetrasodium glutamate diacetate in an amount of about 0.1% by weight, and poloxamer 407 in an amount of about 22-24% by weight; (iii) sodium polymetaphosphate in an amount of about 0.25% by weight, tetrasodium glutamate diacetate in an amount of about 0.25% by weight, and poloxamer 407 in an amount of about 22-24% by weight; (iv) sodium polymetaphosphate in an amount of about 0.5% by weight, tetrasodium glutamate diacetate in an amount of about 0.15% by weight, and poloxamer 407 in an amount of about 22.5-25% by weight; (v) sodium polymetaphosphate in an amount of about 0.5% by weight, tetrasodium glutamate diacetate in an amount of about 0.15% by weight, and poloxamer 407 in an amount of about 18-21% by weight; (vi) sodium polymetaphosphate in an amount of about 0.25% by weight, tetrasodium glutamate diacetate in an amount of about 0.25% by weight, and poloxamer 407 in an amount of about 18-21% by weight; or (vii) sodium polymetaphosphate in an amount of about 0.2% by weight, tetrasodium glutamate diacetate in an amount of about 0.5% by weight, and poloxamer 407 in an amount of about 18-21% by weight.

In another particular such aspect, the composition disclosed herein is a crosslinked polymer-based composition, i.e., a composition according to any one of the embodiments above, which further comprises a water insoluble biodegradable or bioerodible pharmaceutically acceptable crosslinked polymer, and a plasticizer, wherein the amount of said crosslinked polymer in said composition is from about 50% to about 80%, e.g., 50-55%, 55-60%, 60-65%, 65-70%, 70-75%, or 75-80%, by weight, the amount of said plasticizer in said composition is from about 8% to about 13%, e.g., 8-8.5%, 8.5-9%, 9-9.5%, 9.5-10%, 10-10.5%, 10.5-11%, 11-11.5%, 11.5-12%, 12-12.5%, or 12.5-13%, by weight, the amount of said polyphosphate or salt thereof in said composition is from about 0.5% to about 25%, preferably from about 4% to about 25%, e.g., 4-4.4%, 4.4-4.8%, 4.8-5.2%, 5.2-5.6%, 5.6-6%, 6-6.4%, 6.4-6.8%, 6.8-7.2%, 7.2-7.6%, 7.6-8%, 8-8.4%, 8.4-8.8%, 8.8-9.2%, 9.2-9.6%, 9.6-10%, 10-10.4%, 10.4-10.8%, 10.8-11.2%, 11.2-11.6%, 11.6-12%, 12-12.4%, 12.4-12.8%, 12.8-13.2%, 13.2-13.6%, 13.6-14%, 14-14.4%, 14.4-14.8%, 14.8-15.2%, 15.2-15.6%, 15.6-16%, 16-16.4%, 16.4-16.8%, 16.8-17.2%, 17.2-17.6%, 17.6-18%, 18-18.4%, 18.4-18.8%, 18.8-19.2%, 19.2-19.6%, 19.6-20%, 20-20.4%, 20.4-20.8%, 20.8-21.2%, 21.2-21.6%, 21.6-22%, 22-22.4%, 22.4-22.8%, 22.8-23.2%, 23.2-23.6%, 23.6-24%, 24-24.4%, or 24.4-25.0%, by weight, and the amount of said amino acid N,N-diacetic acid or salt thereof in said composition is from about 0.5% to about 10%, preferably from about 0.6% to about 8%, e.g., 0.6-1%, 1-1.4%, 1.4-1.8%, 1.8-2.2%, 2.2-2.6%, 2.6-3%, 3-3.4%, 3.4-3.8%, 3.8-4.2%, 4.2-4.6%, 4.6-5%, 5-5.4%, 5.4-5.8%, 5.8-6.2%, 6.2-6.6%, 6.6-7%, 7-7.4%, or 7.4-8%, by weight, wherein said composition being in a solid dosage form, and upon contact with an aqueous fluid, said composition adsorbs said fluid and consequently swells, and then degrades and releases said polyphosphate or salt thereof and said amino acid N,N-diacetic acid or salt thereof in a sustained release manner.

In certain embodiments, the composition disclosed herein is a crosslinked polymer-based composition, wherein the water insoluble biodegradable or bioerodible pharmaceutically acceptable crosslinked polymer is polylactide (PLA), polyglycolide (PGA), poly(lactic-co-glycolic acid) (PLGA), chitosan oligosaccharide, dextran, starch, alginic acid, hyaluronic acid, carrageenan, hydroxyethylcellulose, carboxymethylcellulose, or a combination thereof.

In certain embodiments, the composition disclosed herein is a crosslinked polymer-based composition, wherein the water insoluble biodegradable or bioerodible pharmaceutically acceptable crosslinked polymer is a protein, more specifically a structural protein. Examples of proteins for use in such compositions include, without being limited to, gelatin optionally hydrolyzed; collagen; an albumin such as serum albumin, milk albumin, or soy albumin; an enzyme such as papain, or chymotrypsin; a serum protein such as fibrinogen; or a combination thereof. The present invention does not require the use of proteins having a specific level of purity, and thus proteins of any grade of purity may be employed. It is, however, preferable to employ proteins having a high degree of purity, and especially a defined (i.e., specifiable) composition, since the use of such proteins increases the degree with which the release of the active agents may be controlled. Thus, it is more preferable to employ a degradation product of proteins such as gelatin (a degradation product of collagen). In particular such embodiments, the polymer comprised within the crosslinked polymer-based composition disclosed herein is thus gelatin, more specifically pharmaceutical grade gelatin, preferably fully or partially hydrolyzed, e.g., bovine source gelatin or a hydrolyzed, e.g., enzymatically hydrolyzed, bovine source gelatin.

The water insoluble biodegradable or bioerodible pharmaceutically acceptable polymer comprised within the composition disclosed herein is crosslinked to an extent that is sufficient to render said polymer insoluble but insufficient to prevent the release of the therapeutic agents from the composition, upon degradation at the treatment site.

In certain embodiments, the composition disclosed herein is a crosslinked polymer-based composition, wherein the plasticizer is a phthalate ester (also termed phthalate), i.e., an ester of phthalic acid, a phosphate ester (also termed organophosphate), i.e., an ester of phosphoric acid, glycerin, or sorbitol. In particular such embodiments, said plasticizer is glycerin.

In certain embodiments, the composition disclosed herein is a crosslinked polymer-based composition, wherein the ratio between said crosslinked polymer and said plasticizer is from about 2:1 to about 10:1, e.g., about 2:1, about 2.5:1, about 3:1, about 3.5:1, about 4:1, about 4.5:1, about 5:1, about 5.5:1, about 6:1, about 6.5:1, about 7:1, about 7.5:1, about 8:1, about 8.5:1, about 9:1, about 9.5:1, or about 10:1, respectively, by weight.

The polymer comprised within the crosslinked polymer-based composition disclosed herein had been crosslinked by any chemical or physical method known in the art, e.g., by a cross-linking agent; an enzyme such as a transglutaminase, tyrosinase, and horseradish peroxidase; or a physical method such as dehydrothermal- and ultraviolet radiation treatment.

In certain embodiments, said polymer had been crosslinked by a cross-linking agent. Examples of cross-linking agents include, without limiting, an aldehyde such as glutaraldehyde and formaldehyde, a carbodiimide such as 1-ethyl-3-(3-dimethylaminopropyl) carbodiimide (EDC), genipin (methyl(1R,4aS,7aS)-1-hydroxy-7-(hydroxymethyl)-1,4a,5,7a-tetrahydroxyxlopenta[c]pyran-4-carboxylate), aluminum, chromium, titanium zirconium, bisdiazobenzidine, phenol 2,4-disulfonyl chloride, 1,5-difluoro-2,4-dinitrobenzene, urea, 3,6-bis(mercurimethyl)-dioxane urea, dimethyl adipimidate, and N,N′-ethylene-bis-(iodo-acetamide). In particular such embodiments, said polymer had been crosslinked by an aldehyde, preferably glutaraldehyde.

In certain embodiments, the composition disclosed herein is a crosslinked polymer-based composition, wherein the polymer is gelatin, preferably fully or partially hydrolyzed; the plasticizer is glycerin; the ratio between said crosslinked polymer and said plasticizer is from about 2:1 to about 10:1, e.g., about 2:1, about 2.5:1, about 3:1, about 3.5:1, about 4:1, about 4.5:1, about 5:1, about 5.5:1, about 6:1, about 6.5:1, about 7:1, about 7.5:1, about 8:1, about 8.5:1, about 9:1, about 9.5:1, or about 10:1, preferably about 6:1, respectively, by weight; and said polymer had been crosslinked by glutaraldehyde. In particular such embodiments, said polyphosphate is polymetaphosphate or a salt thereof such as sodium polymetaphosphate, and said amino acid N,N-diacetic acid is GLDA or a salt thereof such as tetrasodium glutamate diacetate, wherein said polymetaphosphate represents a compound of the formula (NaPO₃)ₙ wherein n is an integer of at least 2 and up to, e.g., 100, or a mixture thereof, comprising, consisting of, or essentially consisting of, hexametaphosphate. More particular such compositions are those comprising sodium polymetaphosphate in an amount of from about 4% to about 18%, e.g., 4-4.4%, 4.4-4.8%, 4.8-5.2%, 5.2-5.6%, 5.6-6%, 6-6.4%, 6.4-6.8%, 6.8-7.2%, 7.2-7.6%, 7.6-8%, 8-8.4%, 8.4-8.8%, 8.8-9.2%, 9.2-9.6%, 9.6-10%, 10-10.4%, 10.4-10.8%, 10.8-11.2%, 11.2-11.6%, 11.6-12%, 12-12.4%, 12.4-12.8%, 12.8-13.2%, 13.2-13.6%, 13.6-14%, 14-14.4%, 14.4-14.8%, 14.8-15.2%, 15.2-15.6%, 15.6-16%, 16-16.4%, 16.4-16.8%, 16.8-17.2%, 17.2-17.6%, or 17.6-18%, by weight; tetrasodium glutamate diacetate in an amount of from about 0.6% to about 5%, e.g., 0.6-1%, 1-1.4%, 1.4-1.8%, 1.8-2.2%, 2.2-2.6%, 2.6-3%, 3-3.4%, 3.4-3.8%, 3.8-4.2%, 4.2-4.6%, or 4.6-5%, by weight, wherein each specific combination of said two ingredients represents a separate embodiment.

In specific embodiments, the composition disclosed herein is a crosslinked polymer-based composition comprising: (i) crosslinked hydrolyzed gelatin in an amount of about 78%, glycerin in an amount of about 12%, sodium polymetaphosphate in an amount of about 4.8% by weight, and tetrasodium glutamate diacetate in an amount of about 4.8% by weight; (ii) crosslinked hydrolyzed gelatin in an amount of about 78%, glycerin in an amount of about 12%, sodium polymetaphosphate in an amount of about 7.2% by weight, and tetrasodium glutamate diacetate in an amount of about 2.4% by weight; (iii) crosslinked hydrolyzed gelatin in an amount of about 76%, glycerin in an amount of about 12%, sodium polymetaphosphate in an amount of about 9.4% by weight, and tetrasodium glutamate diacetate in an amount of about 2.4% by weight; or (iv) crosslinked hydrolyzed gelatin in an amount of about 70%, glycerin in an amount of about 11%, sodium polymetaphosphate in an amount of about 17.2% by weight, and tetrasodium glutamate diacetate in an amount of about 2.2% by weight.

In certain embodiments, the composition disclosed herein is a crosslinked polymer-based composition according to any one of the embodiments above, wherein said composition has a dissolution profile in water, at room temperature, whereby 30%-70%, e.g., about 35%-65%, about 40%-60%, about 45%-55%, or about 50%, preferably about 50%-70%, of said polyphosphate or salt thereof, and/or 30%-70%, e.g., about 35%-65%, about 40%-60%, about 45%-55%, or about 50%, preferably about 50%-70%, of said amino acid N,N-diacetic acid or salt thereof, are released over the first 2 hours. Yet, it should be clear that such compositions may have a dissolution profile in water, at room temperature, whereby identical, similar, or substantially different percentages (within the range recited above) of said polyphosphate or salt thereof and said amino acid N,N-diacetic acid or salt thereof are released over the first 2 hours.

The crosslinked polymer-based composition of the present invention is in fact a solid dosage form, e.g., in the form of a snippet (also referred to as chip). In certain embodiments, said solid dosage form is an essentially two-(or practically flat, although not necessarily uniform, three-) dimensional solid implant adapted for implantation in a periodontal pocket, gingival pocket, or pocket resulting from peri-implantitis. Such a solid implant may have different shapes such as triangles and circles, and may be, e.g., from about 3 to about 10 mm in length, from about 1 to about 5 mm in width, and from about 0.01 to about 2 mm in thickness; or from about 2 to about 6 mm in diameter, and from about 0.01 to about 2 mm in thickness. In particular embodiments, the solid implant has a circle shape having diameter and thickness as defined above, that is lighter/thinner in the circumference.

Periodontal disease, also known as gum disease, is a set of inflammatory conditions affecting the tissues surrounding the teeth. In its early stage, called gingivitis, the gums become swollen, red, and may bleed. Later, in its more serious form called periodontitis, the gums can pull away from the tooth, bone may be lost, and the teeth may loosen or fall out. Periodontal disease is generally due to bacteria in the mouth infecting the tissue around the teeth. Risk factors include smoking, diabetes, HIV/AIDS, family history, and certain medications.

Periodontitis is a widespread disease characterized by inflammation-induced progressive damage to the tooth-supporting structures until tooth loss occurs. The regeneration of lost and/or damaged support tissue in the periodontium, including the alveolar bone, periodontal ligament, and cementum, is the purpose of periodontal regenerative therapy and might effectively reduce periodontitis-caused tooth loss.

Gingival recession, also known as receding gums, is the exposure in the roots of the teeth caused by a loss of gum tissue and/or retraction of the gingival margin from the crown of the teeth. Gum recession is a common problem in adults over the age of 40, but it may also occur earlier and even from the age of a teenager. Gingival recession may exist with or without concomitant decrease in crown-to-root ratio (recession of alveolar bone).

In dental cavities, bacteria reside in biofilm and are hard to remove by toothbrushing. Therefore, the only way to clear the biofilm is to cut the infected site (using a dental bur) and put a filling. If the cavity and the biofilm is close to the dental pulp, this may cause irreversible pulpitis and the need for root canal treatment.

The compositions of the present invention regardless of their specific formulation, i.e., whether formulated as a poloxamer copolymer-based composition or crosslinked polymer-based composition, are useful, e.g., as dental compositions, more specifically for removing microbial biofilm from, or inhibiting or disrupting microbial biofilm formation in, a periodontal pocket, gingival pocket, or pocket resulting from peri-implantitis. In addition, the poloxamer copolymer-based compositions may be used for removing microbial biofilm from, or inhibiting or disrupting microbial biofilm formation in, caries associated with dental cavities or the root canal system; as well as for removing microbial biofilm from, or inhibiting or disrupting microbial biofilm formation on, an orthodontic device such as orthodontic-brace, aligner, extender and bridge.

The dental compositions disclosed are formulated for topical administration/application, and aimed at releasing the therapeutic agents, more specifically the polyphosphate and the chelating agent, i.e., amino acid N,N-diacetic acid or salt thereof, in a periodontal pocket, gingival pocket, pocket resulting from peri-implantitis, or caries associated with dental cavities or the root canal system in a sustained (prolonged) release manner, i.e., a manner aimed at maintaining administration of the therapeutic agents for a specific period of time, e.g., several hours and up to several days, so as to remove microbial biofilm, or inhibit or disrupt microbial biofilm formation, within said pocket or said caries associated with dental cavities or the root canal system.

Application of the composition in a periodontal pocket, gingival pocket, pocket resulting from peri-implantitis, or caries associated with dental cavities or the root canal system, may be carried out using any suitable delivery mean, e.g., a syringe, an applicator, or a dental device capable of delivering a liquid or semi-liquid (e.g., gel) composition into the mouth cavity, and specifically into a periodontal pocket, gingival pocket, pocket resulting from peri-implantitis, or caries associated with dental cavities or the root canal system.

The release profile of the active agents from the dental composition may vary depending on the specific composition (in terms of ingredients and percentage of each one of those ingredients) of said composition and may further be affected by the specific conditions in the mouth of the subject treated. Furthermore, a release profile from a particular composition in gingival crevicular fluid (GCF) might be different than in water.

The compositions of the present invention may be prepared by any suitable techniques, e.g., as described in Remington: The Science and Practice of Pharmacy, 19th Ed., 1995. The compositions can be prepared, e.g., by uniformly and intimately bringing the active agents into association with a liquid carrier, a finely divided solid carrier, or both, and then, if necessary, shaping the product into the desired formulation. Particular procedures for the preparation of poloxamer copolymer-based composition and crosslinked polymer-based composition are disclosed in the Experimental section hereinafter. The compositions disclosed may further include pharmaceutically acceptable fillers, carriers, diluents or adjuvants, as well as other inert ingredients and excipients. Yet, particular such compositions are free of a fluoride such as stannous fluoride, amine fluoride, sodium fluoride, and calcium fluoride, and are preferably free of hydrophobic ingredients.

In another aspect, the present invention relates to a method for removing microbial biofilm from, or inhibiting or disrupting microbial biofilm formation in, a periodontal pocket, gingival pocket, pocket resulting from peri-implantitis, or caries associated with dental cavities or the root canal system, in a subject in need thereof, comprising administering into said periodontal pocket, gingival pocket, pocket resulting from peri-implantitis, or caries associated with dental cavities or the root canal system, a composition as referred to above, e.g., a poloxamer copolymer-based composition or a crosslinked polymer-based composition, as defined in any one of the embodiments above, to thereby release said polyphosphate or salt thereof and said amino acid N,N-diacetic acid or salt thereof in said periodontal pocket, gingival pocket, pocket resulting from peri-implantitis, or caries associated with dental cavities or the root canal system, in a sustained release manner. In certain embodiments, the method disclosed is for removing microbial biofilm from, or inhibiting or disrupting microbial biofilm formation in, a periodontal pocket, gingival pocket, or pocket resulting from peri-implantitis, and comprises (a) topically administering into said periodontal pocket, gingival pocket, or pocket resulting from peri-implantitis, a poloxamer copolymer-based composition according to any one of the embodiments above; or (b) implanting in said periodontal pocket, gingival pocket, or pocket resulting from peri-implantitis, a crosslinked polymer-based composition according to any one of the embodiments above, to thereby release said polyphosphate or salt thereof and said amino acid N,N-diacetic acid or salt thereof in said periodontal pocket, gingival pocket, or pocket resulting from peri-implantitis in a prolonged release manner. In other embodiments, the method disclosed is for removing microbial biofilm from, or inhibiting or disrupting microbial biofilm formation in, caries associated with dental cavities or the root canal system, and comprises (a) topically administering into said caries associated with dental cavities or the root canal system a poloxamer copolymer-based composition according to any one of the embodiments above, to thereby release said polyphosphate or salt thereof and said amino acid N,N-diacetic acid or salt thereof in said caries associated with dental cavities or the root canal system in a prolonged release manner.

The term “subject” as used herein refers to any mammal, e.g., a human, non-human primate, horse, ferret, dog, cat, cow, and goat. In a preferred embodiment, the term “subject” denotes a human, i.e., an individual.

In a further aspect, the present invention relates to a method for removing microbial biofilm from, or inhibiting or disrupting microbial biofilm formation on, an orthodontic device such as orthodontic-brace, aligner, extender and bridge, comprising administering onto said orthodontic device a composition as referred to above, e.g., a poloxamer copolymer-based composition as defined in any one of the embodiments above, to thereby release said polyphosphate or salt thereof and said amino acid N,N-diacetic acid or salt thereof on said orthodontic device in a sustained release manner.

The poloxamer copolymer-based composition of the present invention, as defined in any one of the embodiments above, is in the form of a liquid at room temperature and/or under refrigerated conditions, and upon warming to body temperature solidifies into a viscous gel. This composition may thus be packed, e.g., in a vial, or alternatively in a suitable sealed syringe, wherein the amount of liquid composition in said syringe is sufficient for treating either a sole site (i.e., periodontal pocket, gingival pocket, or pocket resulting from peri-implantitis) or a varying number of sites in the subject.

The sealed syringe may be equipped with a blunt needle, suitable for applying, i.e., topically administering, said composition into, e.g., a periodontal pocket, gingival pocket, or pocket resulting from peri-implantitis, wherein the amount of the composition in the syringe is sufficient for applying into a sole site or more. Such a syringe may be equipped with 25G needle or tip for optimal injection; however, smaller or larger gauge can be used as well. The syringe is best operated at either ambient or below ambient temperature where the viscosity is low enough to allow precise and controlled delivery without exerting excessive pressure. At this temperature, a physician can deliver the right amount of composition directly to the targeted site, where it will turn into gel that will adhere and stay inside the target site. Upon gelation, the highly viscous structure prevents leakage to the surrounding tissue, and controls the release of the therapeutic agents, i.e., the polyphosphate and the chelating agent, in a sustained manner. Alternatively, the liquid composition may be applied into the pocket using an applicator.

In still another aspect, the present invention provides a kit comprising a poloxamer copolymer-based composition as defined in any one of the embodiments above, and a delivery mean, e.g., a syringe or an applicator, for topically administering or applying said composition into a periodontal pocket, gingival pocket, pocket resulting from peri-implantitis, or caries associated with dental cavities or the root canal system.

The delivery mean included in the kit disclosed herein may be any mean capable of administering or applying a predetermined amount of a liquid poloxamer copolymer-based composition as defined herein to a periodontal pocket, gingival pocket, pocket resulting from peri-implantitis, or caries associated with dental cavities or the root canal system in a subject to be treated, e.g., an applicator or a syringe optionally with a blunt needle. In particular embodiments, the kit of the invention comprises a syringe with a blunt needle, capable of administering one or more doses of a liquid poloxamer copolymer-based composition as defined herein to a periodontal pocket, gingival pocket, pocket resulting from peri-implantitis, or caries associated with dental cavities or the root canal system.

In yet another aspect, the present invention provides a kit comprising more than one, i.e., at least 2, 4, 6, 8, 10, 12, or more, solid dosage form made of a crosslinked polymer-based composition as defined in any one of the embodiments above, each being an essentially three-dimensional solid implant adapted for implantation in a periodontal pocket, gingival pocket, or pocket resulting from peri-implantitis.

In still a further aspect, the present invention provides a poloxamer copolymer-based composition as defined in any one of the embodiments above, per se, i.e., a composition comprising at least one polyphosphate or a salt thereof, an amino acid N,N-diacetic acid or a salt thereof, and a non-biodegradable thermosensitive pharmaceutically acceptable poloxamer copolymer, wherein the amount of said poloxamer copolymer in said composition is from about 17% to about 27% by weight, the amount of said polyphosphate or salt thereof in said composition is from about 0.05% to about 3% by weight, and the amount of said amino acid N,N-diacetic acid or salt thereof in said composition is from about 0.025% to about 2% by weight, wherein said composition has a pH in a range of 6-8; and said composition is liquid at room temperature and/or under refrigerated conditions, and upon warming to body temperature, said composition solidifies into a viscous gel. In particular such compositions, said PolyP is a polymetaphosphate and said amino acid N,N-diacetic acid is selected from GLDA, aspartic acid-N,N-diacetic acid, glycine-N,N-diacetic acid, MGDA, serine-N,N-diacetic acid, and alpha- and beta-alanine-N,N-diacetic acid.

In yet a further aspect, the present invention provides a crosslinked polymer-based composition as defined in any one of the embodiments above, per se, i.e., a composition comprising at least one polyphosphate or a salt thereof, an amino acid N,N-diacetic acid or a salt thereof, a water insoluble biodegradable or bioerodible pharmaceutically acceptable crosslinked polymer, and a plasticizer, wherein the amount of said crosslinked polymer in said composition is from about 50% to about 80% by weight, the amount of said plasticizer in said composition is from about 8% to about 13% by weight, the amount of said polyphosphate or salt thereof in said composition is from about 0.5% to about 25%, preferably from about 4% to about 25%, by weight, and the amount of said amino acid N,N-diacetic acid or salt thereof in said composition is from about 0.6% to about 10%, preferably from about 0.5% to about 8% by weight, wherein said composition being in a solid dosage form, and upon contact with an aqueous fluid, said composition adsorbs said fluid and consequently swells, and then degrades and releases said polyphosphate or salt thereof and said amino acid N,N-diacetic acid or salt thereof in a sustained release manner. In particular such compositions, said polyphosphate is a polymetaphosphate and said amino acid N,N-diacetic acid is selected from GLDA, aspartic acid-N,N-diacetic acid, glycine-N,N-diacetic acid, MGDA, serine-N,N-diacetic acid, and alpha- and beta-alanine-N,N-diacetic acid.

Unless otherwise indicated, all numbers expressing quantities of ingredients and so forth used in the present specification are to be understood as being modified in all instances by the term “about”. Accordingly, unless indicated to the contrary, the numerical parameters set forth in this description and attached claims are approximations that may vary by up to plus or minus 10% depending upon the desired properties sought to be obtained by the present invention.

The invention will now be illustrated by the following non-limiting Examples.

### EXAMPLES

### Materials

Poloxamer 407: Synperonic™ PE/F 127-FL-(CQ), Cat #ETK1147/0025/KB16, lot #2001YS3039, Croda; PolyP: Sodium polyphosphates, Glassy, FCC, Cat #SO169, lot #1IH1229, Spectrum; GLDA: Tetrasodium N,N-bis(carboxymethyl)-L-glutamate (ca. 40% in water), Cat #B2135, lot #8XKAN, TCI; gelatin: Byco™ C-PW-(WD), Product code #PR06783/SAMP, lot #970194, Croda; gluteraldehyde: 25% aq. solution, Cat #A17876, Alfa Aesar; glycerin: Cat #100219RK2186, PT Wilmar Nabati Indonesia; deionized water: in-house production.

### Example 1. Poloxamer-Based Preparations

Formulations containing various concentrations of poloxamer and PolyP, with and without GLDA, were prepared according to the following procedure:
1. Preparation of a concentrated stock solution of PolyP (7.5-20% w/w);
2. Preparation of a concentrated neutralized stock solution of GLDA (5-20% w/w) (neutralization of the GLDA stock solution was done with glacial acetic acid, but may be done with any other suitable acid, e.g., citric acid, as well);
3. Preparation of a concentrated poloxamer solution (30% w/w);
4. Combining (i) cold gel and the PolyP concentrated solutions; or (ii) cold gel, the PolyP and the GLDA concentrated solutions, in a ratio calculated to obtain the desired final concentration; and
5. Vortex mixing until homogenous solution is obtained.

Formulations that met initial specifications of appearance, solubility, and desired transition from liquid to gel, were selected for in-vitro studies followed by further selection for in-vivo studies. The gelation of poloxamer-based formulations was tested by placing ˜0.3-1 g of poloxamer preparation (liquid) in an incubator set to 37° C. for 5 and 10 minutes. Thereafter, gelation was visually examined by inverting the tube.

The various formulations prepared and tested are listed in Table 1. Compositions that passed the acceptance criteria are in bold. Increased amount of PolyP resulted in faster gelation (i.e., gelation at a lower temperature) and allowed. Therefore, at PolyP amounts ≥1%, the amount of poloxamer could decrease to 22.5%. Combination of PolyP with GLDA resulted in creation of two phases, i.e., decreased solubility, at the higher dose range tested. Few compositions with PolyP concentration lower than 1% met the specification. The final concentration of the poloxamer may vary in the range of about 17 to about 27%, depending on the specific supplier and even the specific batch of the raw material used.

The phosphate content and stability of a representative composition was determined using the phosphomolybdenum blue (PMB) spectrophotometric assay. The assay determines ortho-phosphate content and therefore, the polyphosphate was first digested to orthophosphate. In sulfuric solution, orthophosphate ions react with molybdate ions to form molybdophosphoric acid. Ascorbic acid reduces this to phosphomolybdenum blue that is determined photometrically. The results of analysis are shown in Table 2 for Formulation #64 as a representative. After 5 months at accelerated 40° C. storage conditions, the PolyP integrity was maintained by the thermosensitive formulation, suggesting shelf life of at least 21 months at room temperature.

The specific composition selected for advance studies is Formulation #74, which comprises poloxamer 407 (25%), PolyP (0.5%), and GLDA (0.1%).

TABLE 1
Formulation matrix for poloxamer copolymer-based PolyP and GLDA preparations

| | | | | | Gelation | |
|---|---|---|---|---|---|---|
| | | | | | (yes/no) | |
| | PX 407 | PolyP | GLDA | | 37° C. | |
| Preparation# | (w/v %) | (w/v %) | (w/v %) | pH | 5/10 min | Comments and state at room temperature |
| 4 | 26.25 | 1.00 | | 7.01 | y/y | Clear |
| 5 | | 2.50 | | 6.83 | y/y | Fast gelation. Two phases observed |
| 6 | | 3.00 | | ND | y/y | Fast gelation. Slight turbidity. |
| | | | | | | Two phases observed |
| 7 | 22.50 | 5.00 | | 6.63 | y/y | Fast gelation. turbid |
| 8 | | 3.75 | | 6.13 | y/y | Fast gelation. turbid |
| 9 | | 3.00 | | 6.71 | y/y | Fast gelation |
| 10 | | 2.50 | | 6.83 | y/y | Fast gelation |
| 11 | | 1.00 | | 6.94 | y/y | Liquid |
| 12 | 20.00 | 2.50 | | 6.75 | n/n | |
| 13 | | 3.00 | | 6.64 | n/n | Liquid. Two phases observed |
| 14 | | 1.00 | | 6.98 | n/n | Liquid |
| 15 | 18.00 | 2.50 | | 6.63 | n/n | Liquid |
| 16 | | 3.00 | | 6.55 | n/n | Liquid |
| 17 | | 1.00 | | 6.91 | n/n | Liquid |
| 26 | 22.50 | 2.50 | 0.63 | ND | ND | Two phases observed |
| 27 | | 1.00 | 1.00 | ND | ND | Two phases observed |
| 28 | 24.00 | 2.50 | 1.00 | ND | y/y | Two phases observed at RT. Gel |
| 29 | | 1.00 | 1.00 | ND | ND | Two phases observed |
| 30 | 26.25 | 1.00 | 1.00 | ND | y/y | Two phases observed. Gel |
| 35 | | | 0.50 | 11.28 | y/y | Soft gel |
| 36 | | | 1.00 | 11.79 | y/y | Soft gel |
| 37 | | 0.50 | 0.50 | 10.51 | y/y | Soft gel |
| 38 | | 2.00 | 0.20 | ND | y/y | Soft gel. Maybe minute liquid phase at |
| | | | | | | bottom |
| 41 | 22.50 | 2.00 | 0.10 | ND | ND | Turbid turns into two clear phases |
| 42 | | 1.00 | 0.50 | ND | ND | Turbid turns into two clear phases, |
| | | | | | | smaller |
| 43 | | 2.00 | 0.50 | ND | ND | Turbid turns into two clear phases, bigger |
| 45 | 24.00 | 1.00 | 0.20 | ND | ND | Turbid turns into two clear phases |
| 47 | | 0.50 | 0.50 | ND | ND | GLDA pre-neutralized. Two clear phases |
| 48 | 26.25 | 0.50 | 0.50 | ND | ND | GLDA pre-neutralized. Two clear phases |
| 49 | | 1.00 | 0.50 | ND | ND | GLDA pre-neutralized. Two clear phases |
| 50 | 24.00 | 0.50 | 0.20 | 7 | y/y | GLDA pre-neutralized. Two clear phases |
| 51 | 26.25 | 0.50 | 0.20 | ND | y/y | GLDA pre-neutralized. Gel |
| 53 | | 0.20 | 0.05 | 7.17 | y/y | GLDA pre-neutralized. Viscus liquid |
| 55 | | 0.20 | 0.50 | 7.12 | y/y | GLDA pre-neutralized. Viscus liquid |
| 57 | | 0.10 | 0.50 | 7.20 | y/y | GLDA pre-neutralized. Viscus liquid |
| 59 | | 0.05 | 0.10 | 7.22 | y/y | GLDA pre-neutralized. Viscus liquid |
| 64 | 24.00 | 0.20 | 0.50 | 6.92 | y/y | GLDA pre-neutralized. Liquid |
| 65 | 25.00 | 0.20 | 0.50 | 7.30 | y/y | GLDA pre-neutralized. Soft gel |
| 70 | 24.00 | 0.20 | | 7.26 | n/y | Liquid |
| 71 | | 0.50 | 0.10 | 6.88 | y/y | GLDA pre-neutralized. Liquid |
| 72 | 22.50 | 0.50 | 0.10 | 6.81 | n/n | GLDA pre-neutralized. Liquid |
| 73 | 24.00 | 0.25 | 0.25 | 6.93 | y/y | GLDA pre-neutralized. Liquid |
| 74 | 25.00 | 0.50 | 0.15 | 6.81 | y/y | GLDA pre-neutralized. Liquid |
| 75 | 24.00 | 0.30 | 0.75 | 6.81 | y/y | GLDA pre-neutralized. Liquid. Slightly |
| | | | | | | opalescence |

TABLE 2
Stability of preparation #64

| | | 25° C./60% | | | | | | |
|---|---|---|---|---|---|---|---|---|
| | | relative | | | | | | |
| | Ini- | humidity | | | | | | |
| Analyte | tial | 3 M | 6 M | 1 M | 2 M | 3 M | 4 M | 5 M |
| Orthophosphate | 0 | 0 | 5.6 | 0 | 4.5 | 0 | 0 | 1.5 |
| (% of | | | | | | | | |
| reference) | | | | | | | | |
| Total P | 98 | 102 | 102 | 106 | 103 | 93 | 93 | 97 |
| (% of nominal) | | | | | | | | |

### Example 2. Chip/Crosslinked Gelatin-Based Preparations

A dosage form of a chip at a thickness of 0.2-0.4 micron is considered optimal for the desired periodontal indications. Crosslinked bovine gelatin was selected as the film forming agent and as the scaffold for carrying polyphosphate. It is a natural biodegradable, non-toxic, mucoadhesive material obtained by controlled hydrolysis of animal skin, bones, and connective tissues. It possesses excellent properties as a vehicle in drug release devices and fulfil various applications in pharmaceutical industries and the biomedical field. Despite all these properties, poor mechanical properties and hygroscopic nature limit its use. Improvements are made with the use of crosslinkers which broaden its applications. The cross-linking has been reported in the literature as an effective and convenient method to modify the release of active ingredient for a longer period and upgrade overall performance of the delivery system. Glutaraldehyde (GA) was selected as the gelatin crosslinker.

The production is done by preparing pre-cut sheet termed “film”, drying it, and then cutting chip size units (snippets). Alternatively, production can be done by “printing” individual chips by dropping pre-determined volume on surface and drying it. Various PolyP amounts, with and without the addition of GLDA, at various amounts and various combinations with PolyP, were prepared and evaluated. Initial acceptance criteria were the appearance, formation of easy to peel homogenous film, without phase separation. The methodology for controlling the film thickness was achieved by using either higher loading volume before drying or more concentrated solutions.

The chip preparation was carried out according to the following steps:
1. Preparation of concentrated solutions of PolyP (20%, density 1.2016 g/ml), GLDA (20%, neutralized) and glutaraldehyde (10%, density 1.0112 gr/ml);
2. Preparation of gelatin and glycerin solutions at the desired concentrations. The ratio of gelatin to glycerin was kept at 6:1, respectively;
3. Addition of the desired amount of 20% PolyP solution to the gelatin solution dropwise while mixing;
4. Addition of the desired amount of neutralized 20% GLDA solution dropwise while mixing;
5. Addition of 10% glutaraldehyde solution dropwise while mixing. The ratio of gelatin to glutaraldehyde was kept at 6:1, respectively;
6. Transfer the solution to a suitable drying plate (so as to obtain, upon drying, a film), or “printing” individual chips by dropping pre-determined volume on a drying plate;
7. Drying (room temperature or 35° C.); and
8. In case of formation of a film in the drying plate, cutting to the desired shape and size of a chip.

The various film preparations are shown in Table 3. Films with relatively high content of both PolyP and GLDA were disqualified due to appearance and separation issues. Films #7, #11, #13, #15, #17, #18 and #19 met the initial acceptance criteria and were further evaluated. The weight and thickness measurements of those films and of Film #1, which served as the control without PolyP, are shown in Tables 4-5. Homogenous weight and thickness for the various compositions tested was obtained. The weights (mg) of all formulations were at a range of 2.9-5.8 mg, average of 3.6-4.7 mg, SD (standard deviation) of 0.3-0.8 and RSD (relative standard deviation) of 6.0-17.4%. The thicknesses were at a range of 157-260 μm, average of 176-220 μm, SD of 13.6-24.7, and RSD of 6.9-13.7%. Evaluation of thicker film preparations is summarized in Table 6. Either increasing the loading volume or concentrating the loading solution resulted in the expected proportional increase in the film thickness while maintaining the initial acceptance criteria for the film.

TABLE 3
Preparation matrix of PolyP-containing crosslinked gelatin films

| | Gelatin | GA | Glycerin | PolyP | GLDA | PolyP | GLDA | |
|---|---|---|---|---|---|---|---|---|
| Film | % in solution | | | | | Nominal % in dry film | | Comments |
| 1 | 6 | 0.5 | 1 | — | — | — | — | Stick to the plate. |
| | | | | | | | | Brown reddish film |
| 3 | | | | 1 | — | 11.8 | — | Brown reddish film |
| 5 | | | | 2 | — | 21.1 | — | |
| 7 | | | | 0.4 | 0.4 | 4.8 | 4.8 | |
| 8 | | | | 0.8 | 0.8 | 8.8 | 8.8 | Disqualified: appearance and |
| 9 | | | | 1.6 | 1.6 | 15.0 | 15.0 | separation issues |
| 10 | | | | 0.8 | 1.6 | 8.1 | 16.2 | |
| 11 | | | | 0.4 | — | 5.1 | — | Brown reddish film |
| 12 | | | | — | 0.4 | — | 5.1 | Disqualified: appearance and |
| | | | | | | | | separation issues |
| 13 | | | | 0.8 | — | 9.6 | — | Brown reddish film |
| 14 | | | | — | 0.8 | — | 9.6 | Disqualified: appearance and |
| | | | | | | | | separation issues |
| 15 | | | | 1.6 | — | 17.6 | — | Brown reddish, |
| | | | | | | | | less uniform, tiny droplets |
| 16 | | | | — | 1.6 | — | 17.6 | Disqualified: appearance and |
| | | | | | | | | separation issues |
| 17 | | | | 0.6 | 0.2 | 7.2 | 2.4 | Brown reddish |
| 18 | | | | 0.8 | 0.2 | 9.4 | 2.4 | Brown reddish |
| 19 | | | | 1.6 | 0.2 | 17.2 | 2.2 | Brown reddish, |
| | | | | | | | | less uniform, tiny droplets |
| 20 | 9 | 0.75 | 1.5 | 0.6 | | 5.1 | — | Deep brown reddish |
| 21 | 12 | 1 | 2 | 0.8 | | 5.1 | — | Deep brown reddish |

TABLE 4
Chip's weight of several compositions (plate loading volume of 20 g)

| | Weight (mg) | | | | | | | |
|---|---|---|---|---|---|---|---|---|
| Formula | 1 | 7 | 11 | 13 | 15 | 17 | 18 | 19 |
| | 3.5 | 3.7 | 3.3 | 3.7 | 4.1 | 2.9 | 3.3 | 3.6 |
| | 3.5 | 4.1 | 3.4 | 3.7 | 4.4 | 3.0 | 3.7 | 3.6 |
| | 3.7 | 4.2 | 3.4 | 3.8 | 4.4 | 3.0 | 3.7 | 3.7 |
| | 3.7 | 4.2 | 3.7 | 3.8 | 4.4 | 3.1 | 4.0 | 3.8 |
| | 3.8 | 4.4 | 4.0 | 3.8 | 4.5 | 3.2 | 4.2 | 4.0 |
| | 3.9 | 4.4 | 4.0 | 3.9 | 4.5 | 3.2 | 4.3 | 4.2 |
| | 4.0 | 4.4 | 4.1 | 4.2 | 4.5 | 3.5 | 4.4 | 4.3 |
| | 4.0 | 4.6 | 4.1 | 4.3 | 4.5 | 3.6 | 4.5 | 4.4 |
| | 4.1 | 4.6 | 4.2 | 4.4 | 4.5 | 3.6 | 4.5 | 4.5 |
| | 4.1 | 4.7 | 4.2 | 4.7 | 4.7 | 3.7 | 4.7 | 5.3 |
| | 4.1 | 4.7 | 4.2 | 5.0 | 4.9 | 3.7 | 4.8 | 5.3 |
| | 4.2 | 4.7 | 4.3 | 5.0 | 4.9 | 4.0 | 4.8 | 5.4 |
| | 4.3 | 4.7 | 4.3 | 5.1 | 5.0 | 4.2 | 5.0 | 5.5 |
| | 4.3 | 4.8 | 4.3 | 5.1 | 5.0 | 4.7 | 5.2 | 5.6 |
| | 4.5 | 5.2 | 4.7 | 5.1 | 5.0 | 4.8 | 5.4 | 5.8 |
| Range (n = 15) | 3.5-4.5 | 3.7-5.2 | 3.3-4.7 | 3.7-5.1 | 4.1-5 | 2.9-4.8 | 3.3-5.4 | 3.6-5.8 |
| Average (n = 15) | 4.0 | 4.7 | 4.0 | 4.4 | 4.6 | 3.6 | 4.4 | 4.6 |
| SD | 0.3 | 0.4 | 0.4 | 0.6 | 0.3 | 0.6 | 0.6 | 0.8 |
| RSD | 7.4 | 7.6 | 9.9 | 13.1 | 6.0 | 16.5 | 13.2 | 17.4 |

TABLE 5
Chip's thickness of several compositions (plate loading volume of 20 g)

| | Thickness (μm) | | | | | | | |
|---|---|---|---|---|---|---|---|---|
| Formula | 1 | 7 | 11 | 13 | 15 | 17 | 18 | 19 |
| | 173 | 183 | 174 | 193 | 200 | 157 | 169 | 196 |
| | 183 | 212 | 185 | 198 | 204 | 162 | 189 | 199 |
| | 186 | 194 | 188 | 181 | 225 | 162 | 191 | 196 |
| | 194 | 213 | 208 | 187 | 232 | 178 | 207 | 204 |
| | 204 | 206 | 217 | 188 | 199 | 156 | 185 | 199 |
| | 175 | 213 | 217 | 199 | 204 | 160 | 208 | 199 |
| | 198 | 217 | 180 | 222 | 206 | 186 | 226 | 213 |
| | 204 | 222 | 204 | 219 | 218 | 169 | 220 | 197 |
| | 194 | 251 | 216 | 191 | 218 | 177 | 227 | 206 |
| | 199 | 203 | 190 | 222 | 218 | 159 | 224 | 241 |
| | 218 | 212 | 217 | 217 | 228 | 175 | 214 | 252 |
| | 211 | 217 | 215 | 229 | 227 | 162 | 224 | 252 |
| | 207 | 226 | 217 | 223 | 205 | 176 | 217 | 230 |
| | 208 | 230 | 229 | 227 | 221 | 235 | 241 | 253 |
| | 213 | 226 | 206 | 229 | 260 | 227 | 222 | 257 |
| Range (n = 15) | 173-213 | 183-251 | 174-229 | 181-229 | 199-260 | 157-235 | 169-241 | 196-257 |
| Average (n = 15) | 197.8 | 202.0 | 204.2 | 208.3 | 217.7 | 176.1 | 210.9 | 219.6 |
| SD | 13.6 | 15.9 | 16.6 | 17.6 | 16.0 | 24.1 | 19.5 | 24.7 |
| RSD | 6.6 | 7.9 | 8.1 | 8.4 | 7.4 | 13.7 | 9.2 | 11.3 |

TABLE 6
Evaluation of thicker film preparations

| | | | Concentrated × 1.5 | Concentrated × 2 |
|---|---|---|---|---|
| Formulation - Film 11 | 20 g/plate | 40 g/plate | 20 g/plate | 20 g/plate |
| Total max expected film | 1.975 | 3.950 | 2,963 | 3.950 |
| weight after drying (g) | | | | |
| Film weight after drying (g) | 1.721 | 3.281 | 2.467 | 3.328 |
| Film weight (% of expected) | 87 | 83 | 83 | 84 |
| Weight ratio to 20 g/plate | 1.00 | 1.91 | 1.43 | 1.93 |
| Film thickness range (μm) | 150-219 | 298-509 | 274-471 | 404-553 |
| Film thickness mean (μm) | 189 | 397 | 350 | 452 |
| Film thickness SD (μm) | 19.2 | 69.7 | 39.9 | 33.2 |
| Film thickness RSD (%) | 10.1 | 17.6 | 11.4 | 7.4 |
| Thickness ratio to 20 g/plate | 1.00 | 2.09 | 1.85 | 2.38 |

### Example 3. In Vitro Release Studies

Initial release studies were performed at room temperature with deionized water as release medium and at specific release medium volume. Samples were withdrawn at predetermined timepoints and analyzed for PolyP content. The results of in vitro release studies for the films and chips are shown in Table 7 and Table 8, respectively. PolyP was released intact with a burst release of about 51-69% of the nominal amount within two hours. The release profile was similar in all chip compositions. Changing the release medium from water to a simulated gingival fluid and increasing the temperature to 37° C. might affect the release profile.

TABLE 7
In vitro release study (films) of various compositions (film size used:
circle, 0.9 cm²; release temperature: room temperature; release medium:
deionized water; release volume: 15 ml; withdrawal volume: 1.5 ml)

| | PolyP content | GLDA content | Film | Amount released (% of nominal) | | | | | |
|---|---|---|---|---|---|---|---|---|---|
| Film | (nominal dry | (nominal dry | weight | 2 | 4 | 23-24 | 2 | 5 | 7 |
| No. | w/w %) | w/w %) | (mg) | hrs | hrs | hrs | days | days | days |
| 7 | 4.8 | 4.8 | 21.6 | 46.5 | | 70.9 | 69.2 | 66.90 | 74.20 |
| | | | 21.0 | 29.0 | 67.55 | 69.5 | 69.5 | 69.43 | 71.35 |
| | | | Mean | 38 | 68 | 70 | 69 | 68 | 73 |
| | | | SD | 12.4 | — | 1.0 | 0.2 | 1.8 | 2.0 |
| 11B | 5.1 | | 19.8 | 74.4 | | 75.7 | 80.1 | 75.77 | 83.40 |
| | | | 22.2 | 75.7 | 68.57 | 77.3 | 77.3 | 78.16 | 77.70 |
| | | | Mean | 75 | 69 | 76 | 79 | 77 | 81 |
| | | | SD | 0.9 | — | 1.2 | 2.0 | 1.7 | 4.0 |
| 13 | 9.6 | | 19.4 | 79.9 | | 82.9 | 76.5 | 78.22 | 87.54 |
| | | | 23.3 | 73.2 | 73.0 | 73.0 | 73.0 | 75.67 | 73.91 |
| | | | Mean | 77 | 73 | 78 | 75 | 77 | 81 |
| | | | SD | 4.8 | — | 7.0 | 2.5 | 1.8 | 9.6 |
| 15 | 17.6 | | 22.4 | 47.4 | | 76.0 | 76.7 | 71.41 | 77.75 |
| | | | 23.1 | 70.4 | 70.3 | 70.0 | 70.0 | 71.24 | 70.18 |
| | | | Mean | 59 | 70 | 73 | 73 | 71 | 74 |
| | | | SD | 16.3 | — | 4.2 | 4.8 | 0.1 | 5.4 |
| 17 | 7.2 | 2.4 | 21.4 | 73.1 | | 84.2 | 77.5 | 80.06 | 89.07 |
| | | | 17.2 | 72.6 | 66.5 | 76.8 | 76.8 | 76.95 | 76.31 |
| | | | Mean | 73 | 66 | 80 | 77 | 79 | 83 |
| | | | SD | 0.3 | — | 5.2 | 0.5 | 2.2 | 9.0 |
| 18 | 9.4 | 2.4 | 20.0 | 59.3 | | 82.9 | 78.5 | 77.05 | 87.93 |
| | | | 24.2 | 71.1 | 77.8 | 72.8 | 72.8 | 74.24 | 73.67 |
| | | | Mean | 65 | 78 | 78 | 76 | 76 | 81 |
| | | | SD | 8.4 | — | 7.2 | 4.1 | 2.0 | 10.1 |
| 19 | 17.2 | 2.2 | 22.2 | 42.0 | | 71.0 | 67.9 | 65.53 | 73.88 |
| | | | 21.7 | 66.6 | 67.0 | 67.1 | 67.1 | 67.29 | 67.23 |
| | | | Mean | 54 | 67 | 69 | 67 | 66 | 71 |
| | | | SD | 17.4 | — | 2.8 | 0.6 | 1.2 | 4.7 |

TABLE 8
In vitro release study (chips) of various compositions (release
temperature: room temperature; release medium: deionized water;
release volume: 2 ml; withdrawal volume: 1.5 ml)

| | PolyP content | GLDA content | Chip | Amount released (% of nominal) | | | | | |
|---|---|---|---|---|---|---|---|---|---|
| Film | (nominal dry | (nominal dry | weight | 2 | | | 24 | | |
| No. | w/w %) | w/w %) | (mg) | hrs | Mean | SD | hrs | Mean | SD |
| 7 | 4.8 | 4.8 | 4.4 | 75 | 69 | 4.7 | 79 | 73 | 5.7 |
| | | | 4.6 | 66 | | | 69 | | |
| | | | 4.7 | 67 | | | 70 | | |
| 11B | 5.1 | | 4.7 | 71 | 66 | 7.7 | 102 | 94 | 13.6 |
| | | | 4.5 | 57 | | | 78 | | |
| | | | 4.6 | 69 | | | 102 | | |
| 13 | 9.6 | | 5.0 | 53 | 58 | 5.6 | 73 | 73 | 4.2 |
| | | | 4.4 | 64 | | | 68 | | |
| | | | 4.0 | 57 | | | 77 | | |
| 15 | 17.6 | | 5.8 | 70 | 59 | 10.0 | 73 | 72 | 3.4 |
| | | | 5.0 | 56 | | | 74 | | |
| | | | 5.1 | 51 | | | 68 | | |
| 17 | 7.2 | 2.4 | 5.0 | 67 | 60 | 8.3 | 82 | 79 | 6.0 |
| | | | 5.4 | 51 | | | 72 | | |
| | | | 4.7 | 64 | | | 83 | | |
| 18 | 9.4 | 2.4 | 4.5 | 57 | 62 | 7.7 | 81 | 84 | 8.5 |
| | | | 3.6 | 57 | | | 77 | | |
| | | | 2.9 | 71 | | | 94 | | |
| 19 | 17.2 | 2.2 | 4.4 | 51 | 51 | 0.8 | 71 | 78 | 11.3 |
| | | | 4.3 | 51 | | | 73 | | |
| | | | 4.5 | 50 | | | 91 | | |

The specific composition selected for advance studies: Formulation #18: PolyP (9.4%), GLDA (2.4%), gelatin (71%) and glycerin (12%). In the solution before drying the ratio gelatin:glycerin is 6:1 and gelatin:glutaraldehyde is 12:1 (it is assumed that the majority of the glutaraldehyde is used for the crosslinking and the glutaraldehyde residues after drying are below the acceptance permitted amount).

### Example 4. Establishing Therapeutic Window for PolyP

Following assay calibration, biofilm made of Streptococcus sanguis, Actinomyces naeslundii, Porphyromonas gingivalis, and Fusobacterium nucleatum was grown on tooth surfaces (hydroxyapatite (HA) disks) or dental implants surfaces (sandblast acid-etched titanium surfaces, SLA).

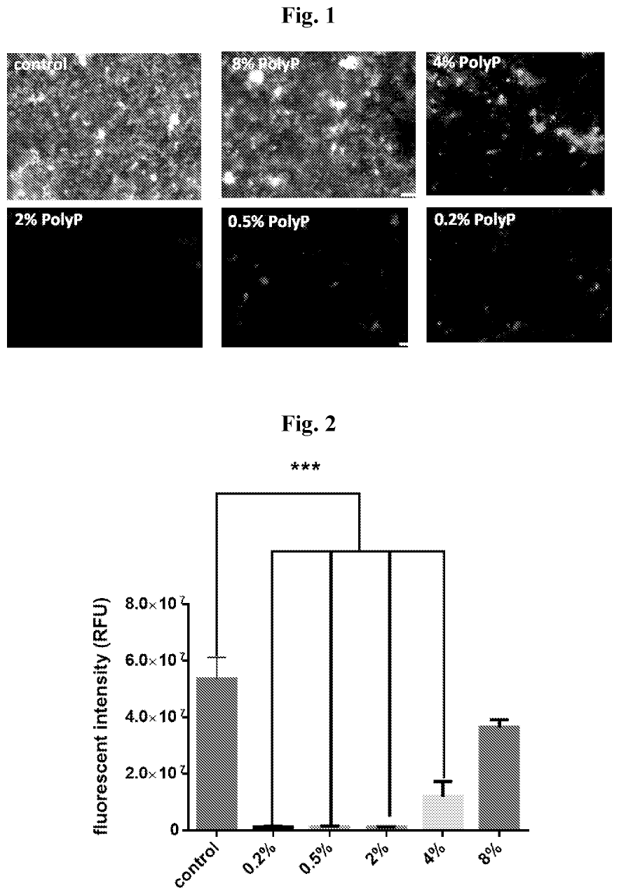

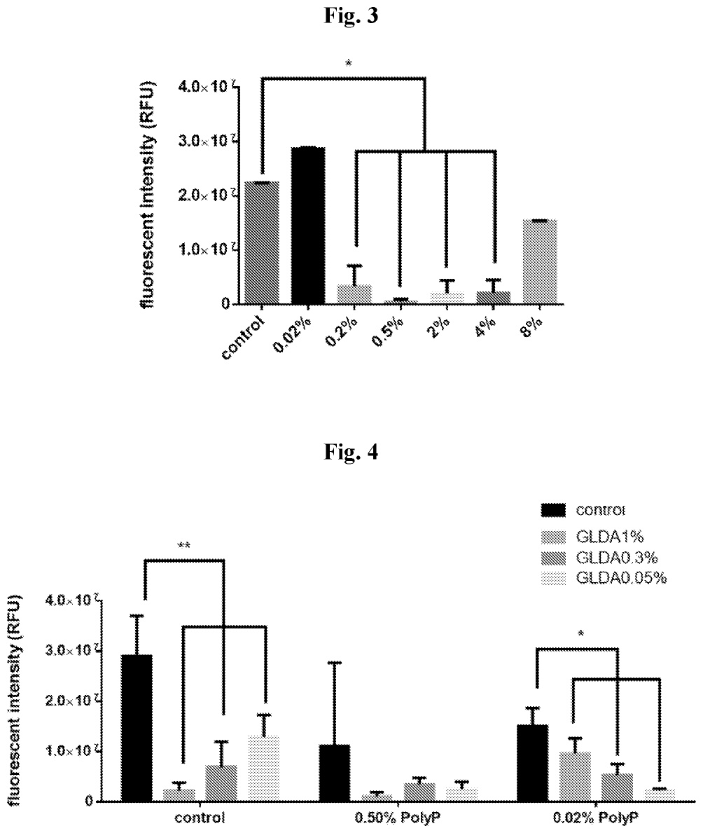

The mature biofilms were washed in saline (control) or saline with PolyP (at 8% to 0.2% w/v) for 45 min at 37° C. The biofilm that remained on the disks was stained with live/dead fluorescent stains and analyzed using a florescence microscope. Results are shown as representative microscopic images (FIG. 1), and as a quantification of the florescence intensity in relative florescence units (RFU) (FIGS. 2-3).

The results show that at 0.2%-4% PolyP in saline, biofilm was significantly reduced compared with control (sham) or higher PolyP concentration (8%) in both tooth and implant surfaces.

### Example 5. Combining Chelating Agents with PolyP

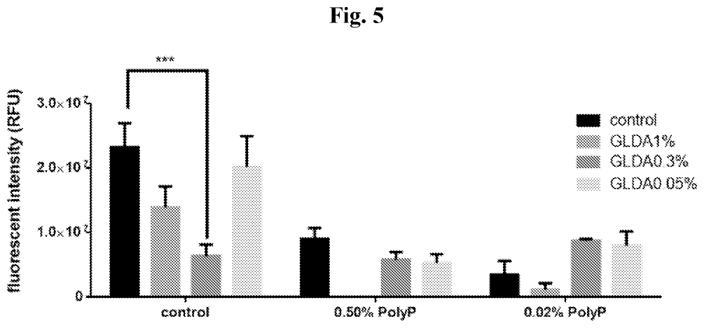

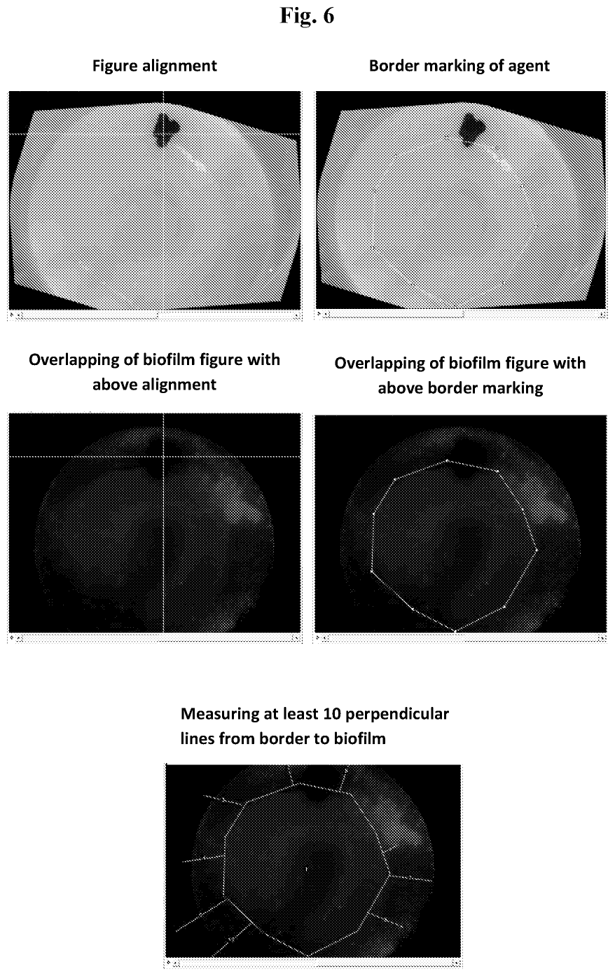

In this experiment, the ability of the chelating agent GLDA to clear biofilm, either alone or in combination with PolyP at 0.02% and 0.5%, was tested. As described in Example 4, biofilm was grown and treated with the different solutions, and the residual biofilm was stained and quantified. The results are shown as a quantification of the florescence intensity in relative florescence units (RFU) (FIGS. 4-5).

The results show that in the case of hydroxyapatite discs (FIG. 4), while GLDA alone show a dose dependent efficacy on biofilm, its combination with PolyP showed a reverse pattern, in which the lowest GLDA concentration combined with PolyP showed the most effective outcome. The same trend was observed with titanium disks (FIG. 5). Overall, the results show that a combination of 0.5% PolyP with low concentration of GLDA results in effective biofilm removal.

### Example 6. pH Changes of PolyP Combinations and the Diffusion Effect of Poloxamer/Biodegradable Film Containing PolyP

While acidic or basic environment in solution breakdown biofilm, it may be harmful for the human tissue at the periodontal pockets. In this experiment we thus measured the pH levels of the GLDA/PolyP combination that were tested in Example 5. As shown in Table 9, GLDA at high concentration is basic while at low levels (0.05%) is neutral; and its combinations with PolyP did not alter pH values.

TABLE 9
pH of various GLDA-PolyP combinations

| Additive | No PolyP | 0.50% PolyP | 0.02% PolyP |
|---|---|---|---|
| GLDA 1% | 11.5 | 11.5 | 11.8 |
| GLDA 0.3% | 9.6 | 9 | 9.2 |
| GLDA 0.05% | 7 | 7 | 7 |

Next, we incorporated the GLDA with PolyP in two carriers—a gel like that solidify in oral conditions (poloxamer) and a biodegradable film. Those compositions were prepared with neutralized GLDA to avoid the pH effect. In the following experiments (diffusion effect assay) the materials were placed on disks covered with mature biofilm, and the biofilm inhibition effect of said material, i.e., the radius of the biofilm free area (measured in pixel values from the material tested outwards, and named “halo distance” or “diffusion effect”), was then tested.

### Diffusion Effect of Poloxamer Containing PolyP

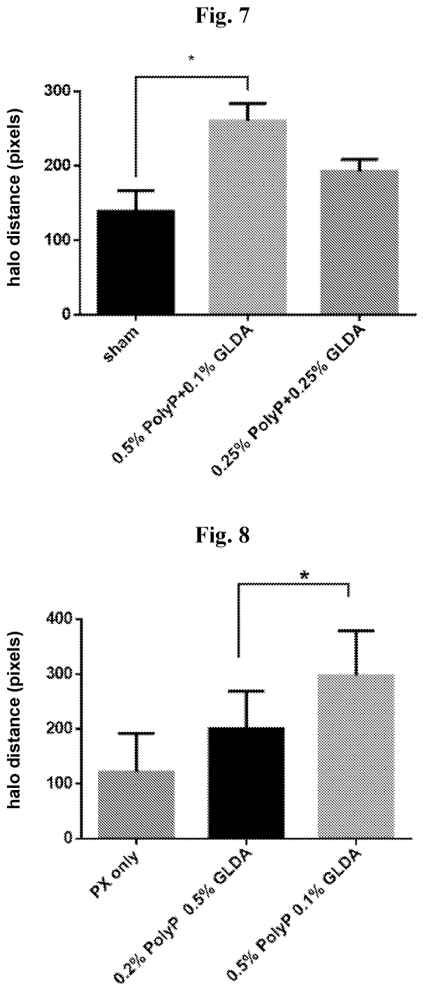

Using the diffusion assay, the effect of poloxamer gels with PolyP and GLDA was measured (presented as halo distance levels). The results, summarized in FIGS. 7-8, show that both tested GLDA/PolyP in poloxamer have a significant diffusion effect.

### Diffusion Effect of Films Containing PolyP

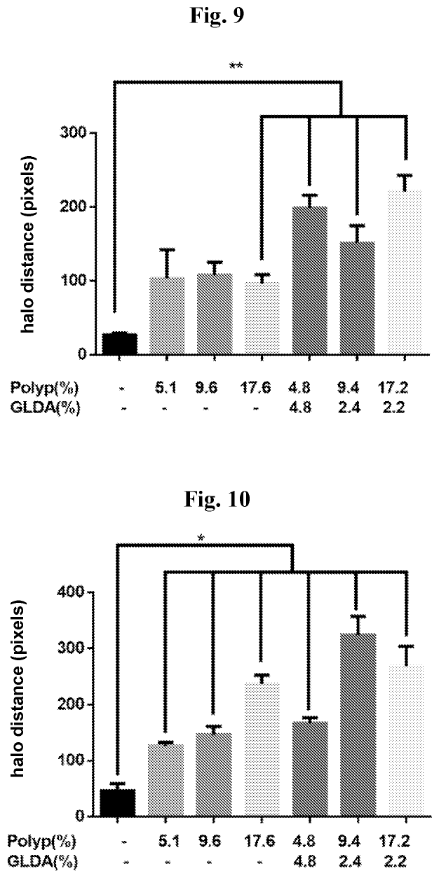

Next, the effect of films was tested at different concentrations of GLDA/PolyP, as described above. The results, summarized in FIGS. 9-10, show that in this case as well, both tested GLDA/PolyP in films have a significant diffusion effect.

### Example 7. Animal Experiments

In this Study, a pig model was used to examine the therapeutic efficacy of the PolyP-GLDA composition, formulated either as a gel or a solid dosage form (film). Four pigs at 18 months of age underwent implant placement between the lower canines. After an osseointegration period of 6 weeks, ligatures of silk 4-0 suture immersed with the same bacteria as in the in vitro biofilm models were ligated around the implants and teeth. After 4 weeks of periodontal/peri-implant pocket formation, one implant and one tooth sites were assigned for the following treatment: (1) gel test treatment; (2) gel sham treatment; (3) film test treatment 4; or (4) film sham treatment.

Prior to the treatment, as well as 8 weeks later, all sites in all animals were sampled for gingival crevicular fluid (GCF) and microbiome analysis.

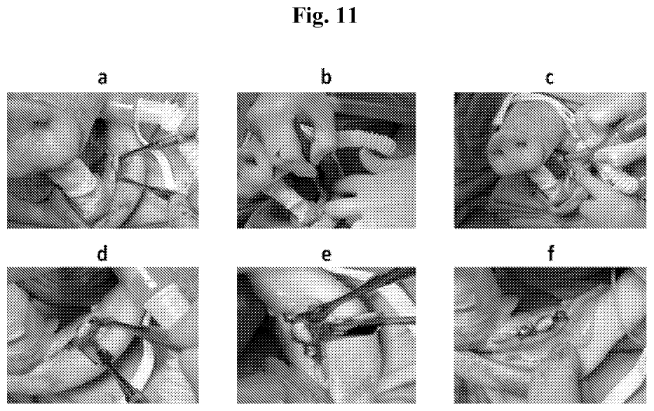

FIG. 11 shows the procedure for establishing a pig model with implants and teeth. In brief, after animal anesthesia, four implants were inserted adjacent to the lower jaw canine and gold-shaded healing caps were screwed to the implants. The steps specifically illustrated are incision in the gums, tissue elevation to expose bone, drilling in the bone and insertion of dental implant, screwing a golden shaded healing cap, and suturing with resorbable string.

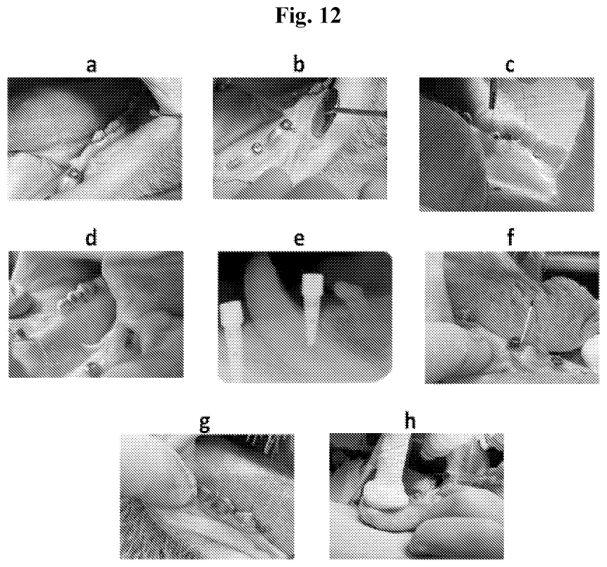

FIGS. 12A-12F shows induction of periodontitis and periimplantitis in the pig model (6 weeks after implant insertion), using infected ligatures (silk strings immersed in the same bacteria used to construct the biofilm in the in vitro models; known as a ligature model). The ligatures were tied around the teeth and implants, and the string was tucked into the physiological gap that exist between the gums and the implant/teeth. 4 weeks from ligature placement, infected pockets (gaps between the gums and implant/teeth) were formed. The silk strings were removed, and specific sites were treated with sham or test gel/film. Prior to treatment, as well as 8 weeks post treatment, the pocket fluids (GCFs) were collected for cytokine and microbiome analysis.

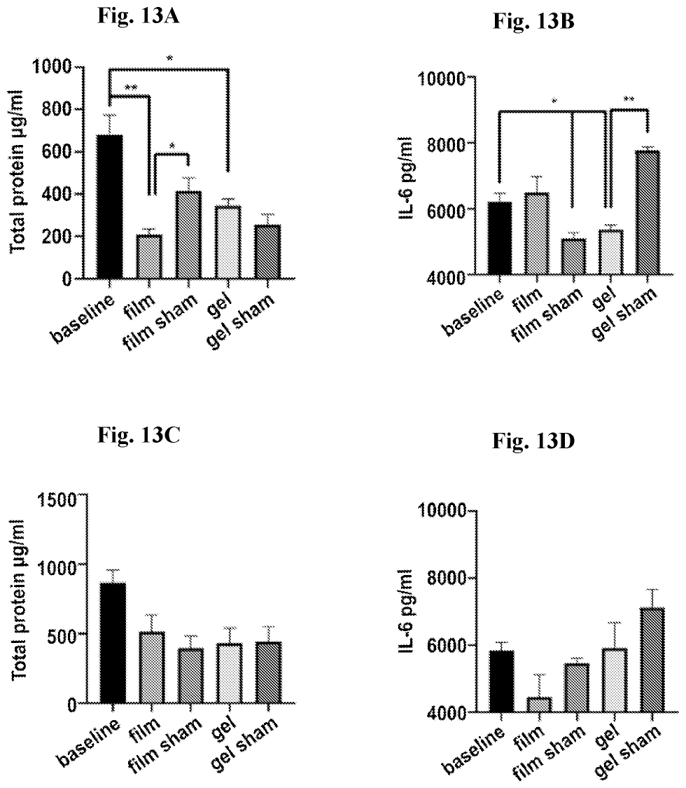

First, we examined inflammation at the pockets, by measuring the inflammatory cytokine IL6 and total GCF protein levels. FIGS. 13A-13D show GCF's total protein and IL6 levels before treatment (immediately after ligature removal) and 8 weeks post treatment. As shown, all treatment reduced total protein levels compared with the baseline, and in the chip groups, the test showed augmented reduced protein levels compared with the sham chip group (P<0.05). IL6 levels showed reduced levels in the film sham and the gel groups (with statistical differences between the gel and the gel sham (P<0.01). In implants, the same pattern was observed albeit with less significant differences, which is reasonable due to low inflammatory response ability around implants due to the lack of the periodontal ligament (PDL) tissue.

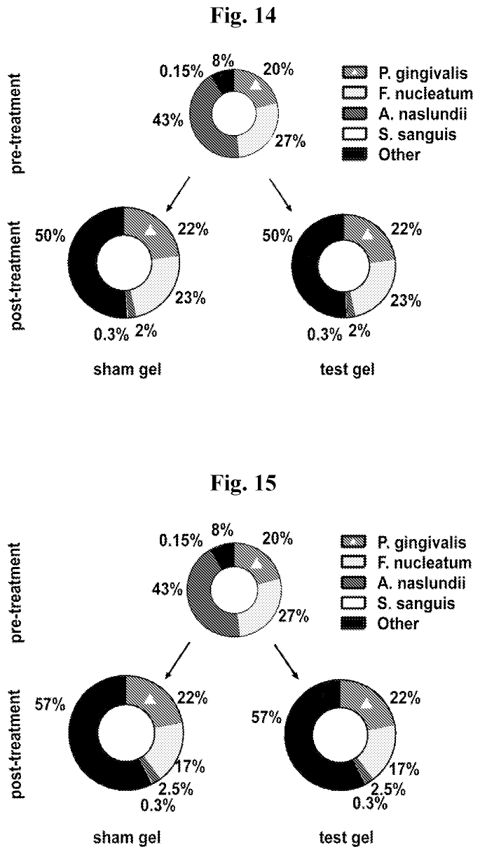

Next, changes in the microbiome were tested. FIG. 14 shows microbiome profile before (pre-treatment) and after treatment with the gel PolyP-GLDA in teeth. The results present the relative abundance (in %) of the perio-pathogens P. gingivalis and F. nucleatum, and the commensal microbes Strep sanguis, A. naslundii and porcine endogenous bacteria (gray). The size of the pie represents the total amount of bacteria (in arbitrary units) in the site. As shown, teeth at baseline (immediately after ligature removal and before treatment) show that half of the bacteria in the biofilm is pathogenic (P. gingivalis and F. nucleatum, versus commensal and porcine endogenous bacteria). Treatment with gels (either sham or test) reduced the proportion of pathogenic bacteria (P. gingivalis and F. nucleatum) and augmented the levels of commensal bacteria (commensal and porcine endogenous bacteria). The gel test also reduced the abundance of biofilm compared with the sham gels (as indicated by the reduction of the pie size between the groups).

Treatment with the films on teeth showed the same pattern with recued dysbiotic biofilm (reduction in P. gingivalis and F. nucleatum proportion in the biofilm) in both groups (test and sham), and with a reduction in biofilm abundance in the test film compared with the sham control, as shown in FIG. 15.

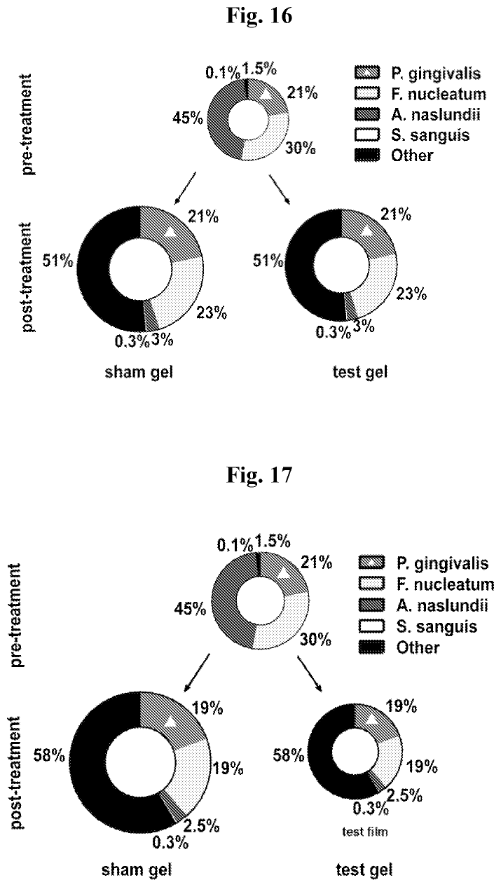

Focusing on implants treated with gel showed similar results to that found on teeth (FIG. 16). The most impressive results were on implants tested with the films, which showed significant reduction in biofilm levels at sites treated with the film test compared with sham control (FIG. 17).

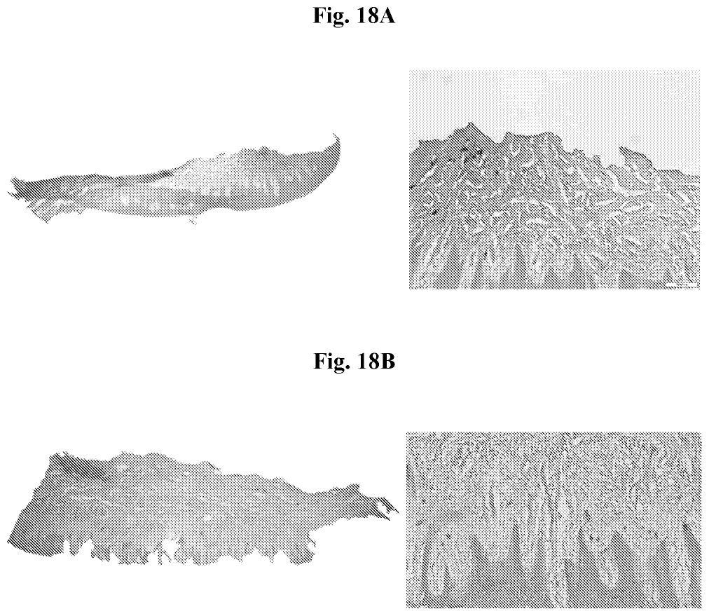

Histological analysis of all sites adjacent to teeth that were treated showed neither pathological phenotype nor evidence of inflammation, indicating healthy gum tissue (FIG. 18).

### REFERENCES

Humphreys, G.; Lee, G. L.; Percival, S. L.; McBain, A. J., Combinatorial activities of ionic silver and sodium hexametaphosphate against microorganisms associated with chronic wounds. Journal of Antimicrobial Chemotherapy, 2011, 66(11), 2556-2561
Hyoju, S. K.; Klabbers, R. E.; Aaron, M.; Krezalek, M. A.; Zaborin, A.; Wiegerinck. M.; Hyuman, N. H.; Zzborina, O.; Van Goor, H.; Alverdy, J. C., Oral polyphosphate suppresses bacterial collagenase production and prevents anastomotic leak due to Serratia marcescens and Pseudomonas aeruginosa. Ann. Surg., 2018, 267(6), 1112-1118

1-32. (canceled)

33. A method for removing microbial biofilm from, or inhibiting or disrupting microbial biofilm formation in, a periodontal pocket, gingival pocket, pocket resulting from peri-implantitis, or caries associated with dental cavities or a root canal system, in an individual in need thereof, comprising administering into said periodontal pocket, gingival pocket, pocket resulting from peri-implantitis, or caries associated with dental cavities or the root canal system, a composition comprising, as an active agent, at least one polyphosphate (PolyP) or a salt thereof, and an amino acid N,N-diacetic acid or a salt thereof, to thereby release said polyphosphate or salt thereof and said amino acid N,N-diacetic acid or salt thereof in said periodontal pocket, gingival pocket, pocket resulting from peri-implantitis, or caries associated with dental cavities or the root canal system in a sustained release manner.

34-74. (canceled)

75. The method of claim 33, wherein said polyphosphate salt is a salt of an alkali metal such as sodium or potassium, alkaline earth metal such as magnesium or calcium, ammonium, or a mixture thereof.

76. The method of claim 33, wherein said polyphosphate is a polymetaphosphate, optionally comprising hexametaphosphate.

77. The method of claim 33, wherein said polyphosphate salt is sodium polymetaphosphate comprising sodium hexametaphosphate.

78. The method of claim 33, wherein said amino acid N,N-diacetic acid is selected from the group consisting of glutamic acid-N,N-diacetic acid (GLDA), aspartic acid-N,N-diacetic acid, glycine-N,N-diacetic acid, methylglycine-N,N-diacetic acid (MGDA), serine-N,N-diacetic acid, and alpha- and beta-alanine-N,N-diacetic acid

79. The method of claim 33, wherein said composition comprises polymetaphosphate or a salt thereof such as sodium polymetaphosphate, and GLDA or a salt thereof such as tetrasodium glutamate diacetate.

80. The method of claim 33, wherein the ratio between said polyphosphate or salt thereof and said amino acid N,N-diacetic acid or salt thereof in said composition is from about 10:1 to about 1:6, respectively, by weight.

81. The method of claim 33, wherein said composition further comprises a non-biodegradable thermosensitive pharmaceutically acceptable poloxamer copolymer, wherein the amount of said poloxamer copolymer in said composition is from about 17% to about 27% by weight, the amount of said polyphosphate or salt thereof in said composition is from about 0.05% to about 3% by weight, and the amount of said amino acid N,N-diacetic acid or salt thereof in said composition is from about 0.025% to about 2% by weight,
wherein said composition has a pH in a range of 6-8; and said composition is liquid at room temperature and/or under refrigerated conditions, and upon warming to body temperature, said composition solidifies into a viscous gel.

82. The method of claim 81, wherein said poloxamer copolymer is poloxamer 407, poloxamer 188, poloxamer 124, poloxamer 237, poloxamer 338, or a mixture thereof.

83. The method of claim 82, wherein said poloxamer copolymer is poloxamer 407, said polyphosphate is polymetaphosphate or a salt thereof such as sodium polymetaphosphate, and said amino acid N,N-diacetic acid is GLDA or a salt thereof such as tetrasodium glutamate diacetate.

84. The method of claim 83, wherein said composition comprises sodium polymetaphosphate in an amount of from about 0.1% to about 0.8% by weight, tetrasodium glutamate diacetate in an amount of from about 0.025% to about 0.8% by weight, and poloxamer 407 in an amount of from about 17% to about 26% by weight.

85. The method of claim 84, wherein said composition comprises:
(i) sodium polymetaphosphate in an amount of about 0.2% by weight, tetrasodium glutamate diacetate in an amount of about 0.5% by weight, and poloxamer 407 in an amount of about 22-24% by weight;
(ii) sodium polymetaphosphate in an amount of about 0.5% by weight, tetrasodium glutamate diacetate in an amount of about 0.1% by weight, and poloxamer 407 in an amount of about 22-24% by weight;
(iii) sodium polymetaphosphate in an amount of about 0.25% by weight, tetrasodium glutamate diacetate in an amount of about 0.25% by weight, and poloxamer 407 in an amount of about 22-24% by weight;
(iv) sodium polymetaphosphate in an amount of about 0.5% by weight, tetrasodium glutamate diacetate in an amount of about 0.15% by weight, and poloxamer 407 in an amount of about 22.5-25% by weight;
(v) sodium polymetaphosphate in an amount of about 0.5% by weight, tetrasodium glutamate diacetate in an amount of about 0.15% by weight, and poloxamer 407 in an amount of about 18-21% by weight;
(vi) sodium polymetaphosphate in an amount of about 0.25% by weight, tetrasodium glutamate diacetate in an amount of about 0.25% by weight, and poloxamer 407 in an amount of about 18-21% by weight; or
(vii) sodium polymetaphosphate in an amount of about 0.2% by weight, tetrasodium glutamate diacetate in an amount of about 0.5% by weight, and poloxamer 407 in an amount of about 18-21% by weight.

86. The method of claim 33, wherein said composition further comprises a water insoluble biodegradable or bioerodible pharmaceutically acceptable crosslinked polymer, and a plasticizer, wherein the amount of said crosslinked polymer in said composition is from about 50% to about 80% by weight, the amount of said plasticizer in said composition is from about 8% to about 13% by weight, the amount of said polyphosphate or salt thereof in said composition is from about 0.5% to about 25% by weight, and the amount of said amino acid N,N-diacetic acid or salt thereof in said composition is from about 0.6% to about 10% by weight,
wherein said composition being in a solid dosage form, and upon contact with an aqueous fluid, said composition adsorbs said fluid and consequently swells, and then degrades and releases said polyphosphate or salt thereof and said amino acid N,N-diacetic acid or salt thereof in a sustained release manner.

87. The method of claim 86, wherein:
(i) said polymer is polylactide (PLA), polyglycolide (PGA), poly(lactic-co-glycolic acid) (PLGA), chitosan oligosaccharide, dextran, starch, alginic acid, hyaluronic acid, carrageenan, hydroxyethylcellulose, carboxymethylcellulose, or a combination thereof;
(ii) said polymer is a protein;
(iii) said plasticizer is a phthalate ester, a phosphate ester, glycerin, or sorbitol;
(iv) the ratio between said crosslinked polymer and said plasticizer in said composition is from about 2:1 to about 10:1, respectively, by weight; or
(v) said polymer had been crosslinked by a cross-linking agent; an enzyme such as a transglutaminase, tyrosinase, and horseradish peroxidase; or a physical method such as dehydrothermal- and ultraviolet radiation treatment.

88. The method of claim 87, wherein:
(i) said protein is gelatin optionally hydrolyzed; collagen; an albumin such as serum albumin, milk albumin, or soy albumin; an enzyme such as papain, or chymotrypsin; a serum protein such as fibrinogen; or a combination thereof; or
(ii) wherein said cross-linking agent is an aldehyde such as glutaraldehyde or formaldehyde, a carbodiimide such as 1-ethyl-3-(3-dimethylaminopropyl) carbodiimide (EDC), genipin, aluminum, chromium, titanium, zirconium, bisdiazobenzidine, phenol 2,4-disulfonyl chloride, 1,5-difluoro-2,4-dinitrobenzene, urea, 3,6-bis(mercurimethyl)-dioxane urea, dimethyl adipimidate, or N,N′-ethylene-bis-(iodo-acetamide).

89. The method of claim 86, wherein said polymer is gelatin optionally hydrolyzed; said plasticizer is glycerin; the ratio between said crosslinked polymer and said plasticizer is from about 2:1 to about 10:1, respectively, by weight; and said polymer had been crosslinked by glutaraldehyde.

90. The method of claim 89, wherein said polyphosphate is polymetaphosphate or a salt thereof such as sodium polymetaphosphate, and said amino acid N,N-diacetic acid is GLDA or a salt thereof such as tetrasodium glutamate diacetate.

91. The method of claim 90, wherein said composition comprises sodium polymetaphosphate in an amount of from about 4% to about 18% by weight, and tetrasodium glutamate diacetate in an amount of from about 0.6% to about 5% by weight.

92. The method of claim 91, wherein said composition comprises:
(i) crosslinked hydrolyzed gelatin in an amount of about 78%, glycerin in an amount of about 12%, sodium polymetaphosphate in an amount of about 4.8% by weight, and tetrasodium glutamate diacetate in an amount of about 4.8% by weight;
(ii) crosslinked hydrolyzed gelatin in an amount of about 78%, glycerin in an amount of about 12%, sodium polymetaphosphate in an amount of about 7.2% by weight, and tetrasodium glutamate diacetate in an amount of about 2.4% by weight;
(iii) crosslinked hydrolyzed gelatin in an amount of about 76%, glycerin in an amount of about 12%, sodium polymetaphosphate in an amount of about 9.4% by weight, and tetrasodium glutamate diacetate in an amount of about 2.4% by weight; or
(iv) crosslinked hydrolyzed gelatin in an amount of about 70%, glycerin in an amount of about 11%, sodium polymetaphosphate in an amount of about 17.2% by weight, and tetrasodium glutamate diacetate in an amount of about 2.2% by weight.

93. The method of claim 86, wherein said composition has a dissolution profile in water, at room temperature, whereby 30-70% of said polyphosphate or salt thereof and said amino acid N,N-diacetic acid or salt thereof is released over the first 2 hours.

94. The method of claim 86, wherein said solid dosage form is a flat three-dimensional solid implant adapted for implantation in a periodontal/peri-implant pocket.

95. The method of claim 94, wherein said implant is from about 3 to about 10 mm in length, from about 1 to about 5 mm in width, and from about 0.01 to about 2 mm in thickness; or from about 2 to about 6 mm in diameter, and from about 0.01 to about 2 mm in thickness.

96. The method of claim 33, for removing microbial biofilm from, or inhibiting or disrupting microbial biofilm formation in, a periodontal pocket, gingival pocket, pocket resulting from peri-implantitis, or caries associated with dental cavities or the root canal system,
wherein said composition further comprises a non-biodegradable thermosensitive pharmaceutically acceptable poloxamer copolymer, wherein the amount of said poloxamer copolymer in said composition is from about 17% to about 27% by weight, the amount of said polyphosphate or salt thereof in said composition is from about 0.05% to about 3% by weight, and the amount of said amino acid N,N-diacetic acid or salt thereof in said composition is from about 0.025% to about 2% by weight,
wherein said composition has a pH in a range of 6-8; and said composition is liquid at room temperature and/or under refrigerated conditions, and upon warming to body temperature, said composition solidifies into a viscous gel,
and wherein said composition is topically administered into said periodontal pocket, gingival pocket, pocket resulting from peri-implantitis, or caries associated with dental cavities or the root canal system.

97. The method of claim 33, for removing microbial biofilm from, or inhibiting or disrupting microbial biofilm formation in, a periodontal pocket, gingival pocket, or pocket resulting from peri-implantitis,
wherein said composition further comprises a water insoluble biodegradable or bioerodible pharmaceutically acceptable crosslinked polymer, and a plasticizer, wherein the amount of said crosslinked polymer in said composition is from about 50% to about 80% by weight, the amount of said plasticizer in said composition is from about 8% to about 13% by weight, the amount of said polyphosphate or salt thereof in said composition is from about 0.5% to about 25% by weight, and the amount of said amino acid N,N-diacetic acid or salt thereof in said composition is from about 0.6% to about 10% by weight,
wherein said composition being in a solid dosage form, and upon contact with an aqueous fluid, said composition adsorbs said fluid and consequently swells, and then degrades and releases said polyphosphate or salt thereof and said amino acid N,N-diacetic acid or salt thereof in a sustained release manner,
and wherein said composition is implanted in said periodontal pocket, gingival pocket, or pocket resulting from peri-implantitis.

98. A method for removing microbial biofilm from, or inhibiting or disrupting microbial biofilm formation on, an orthodontic device such as orthodontic brace, aligner, extender and bridge, comprising administering onto said orthodontic device a composition comprising, as active agents, at least one polyphosphate (PolyP) or a salt thereof, and an amino acid N,N-diacetic acid or a salt thereof, to thereby release said polyphosphate or salt thereof and said amino acid N,N-diacetic acid or salt thereof on said orthodontic device in a sustained release manner,
wherein said composition optionally further comprises a non-biodegradable thermosensitive pharmaceutically acceptable poloxamer copolymer, wherein the amount of said poloxamer copolymer in said composition is from about 17% to about 27% by weight, the amount of said polyphosphate or salt thereof in said composition is from about 0.05% to about 3% by weight, and the amount of said amino acid N,N-diacetic acid or salt thereof in said composition is from about 0.025% to about 2% by weight, and wherein said composition has a pH in a range of 6-8; and said composition is liquid at room temperature and/or under refrigerated conditions, and upon warming to body temperature, said composition solidifies into a viscous gel.
